SEQUENCE LISTING (101 sequences)

SEQ ID NO: 1 - protein, 118 aa, Synthetic construct
QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSS

SEQ ID NO: 2 - protein, 107 aa, Synthetic construct
DIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIK

SEQ ID NO: 3 - protein, 116 aa, Synthetic construct
EVQLVESGGGLVQPGRSLKLSCAASGFSFSNYGMAWVRQAATKGLEWVASISTGGGNTYYRDSVKGRFIVSRDNAKNTQYLQMDSLRSEDTATYYCTRHDRGGLYWGQGVMVTVSS

SEQ ID NO: 4 - protein, 112 aa, Synthetic construct
DIVMTQAPLSVSVTPGESASISCRSSKSLLHSNGITYVYWYFQKPGKSPQVLIYRMSNLASGVPDRFSGSGSETDFTLKISRVEAEDVGIYHCGQLLENPYTFGAGTELELK

SEQ ID NO: 5 - protein, 330 aa, Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 107 aa, Synthetic construct [sequence omitted]

SEQ ID NO: 7 - protein, 232 aa, Synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 345 aa, Synthetic construct
MYKDCIESTGDYFLLCDAEGPWGIILESLAILGIVVTILLLLAFLFLMRKIQDCSQWNVLPTQLLFLLSVLGLFGLAFAFIIELNQQTAPVRYFLFGVLFALCFSCLLAHASNLVKLVRGCVSFSWTTILCIAIGCSLLQIIIATEYVTLIMTRGMMFVNMTPCQLNVDFVVLLVYVLFLMALTFFVSKATFCGPCENWKQHGRLIFITVLFSIIIWVVWISMLLRGNPQFQRQPQWDDPVVCIALVTNAWVFLLLYIVPELCILYRSCRQECPLQGNACPVTAYQHSFQVENQELSRARDSDGAEEDVALTSYGTPIQPQTVDPTQECFIPQAKLSPQQDAGGV

SEQ ID NO: 9 - protein, 344 aa, Synthetic construct
MYKDCIESTGDYFLPCDSEGPWGIVLESLAILGIVVTILLLLAFLFLMRKIQDCSQWNVLPTQLLFLLSVLGLFGLAFAFIIQLNQQTAPVRYFLFGVLFALCFSCLLAHASNLVKLVRGRVSFSWTTILCIAIGCSLLQVIIAIEYVTLIMTRGMMFVHMTPYQLNVDFVVLLVYVLFLMALTFFVSKATFCGPCENWKQHGRLIFITVLFSIIIWVVWISMLLRGNPQFQRQPQWDDPVVCIALVTNAWVFLLLYIVPELCILYRSCRQECPSQGHACPVTAYQRSFQVENQELSRARDSDGAEEDVALTSFGTPIQPQTVDPTQECFIPRAKLSPQQDAGV

SEQ ID NO: 10 - protein, 124 aa, Synthetic construct
EVQLQASGGGSVQAGGSRRLSCVASGYAGGIAYMAWFRQTPGKGREGVALIATSGTTSYANSVKGRFTIFRDGADNTVYLEMSSLKPEDTATYYCAAKSRLGWPGLETSQFKYWGQGTQVTVSS

SEQ ID NO: 11 - protein, 120 aa, Synthetic construct
DVQLQASGGGSVQAGESLRLSCAASGYLYMGWFRQAPGKEREGVAAIVTQSGLTGYADSVKGRFTISQDNAENRIYLQMNSLKPEDTAMYYCAAKSRLGWTLSPSAYNYWGRGTQVTVSS

SEQ ID NO: 12 - protein, 118 aa, Synthetic construct
DVQLQASGGGSVQAGGSLRLSCAASGDTANCIGWFRQAPGKGREGVAAIYTGLGNTYYANSAKGRFTIAQDNAKNTVYLQMNSLKPEDSAMYYCAAGGILGRFCSRYFGQGTQVTVSS

SEQ ID NO: 13 - protein, 126 aa, Synthetic construct
EVQLQASGGGSVQAGGSLRLSCVASGFPYSTSRVGWFRQAPGKEREGVATIVPGTGYTYYADSVKGRFTISQDSAKNTVYLQMNSLKPEDTAVYYCAAGDRRLLLNLLAPADYFYWGQGTQVTVSS

SEQ ID NO: 14 - protein, 8 aa, Synthetic construct
GYAGGIAY

SEQ ID NO: 15 - protein, 7 aa, Synthetic construct
IATSGTT

SEQ ID NO: 16 - protein, 18 aa, Synthetic construct
AAKSRLGWPGLETSQFKY

SEQ ID NO: 17 - protein, 4 aa, Synthetic construct
GYLY

SEQ ID NO: 18 - protein, 8 aa, Synthetic construct
IVTQSGLT

SEQ ID NO: 19 - protein, 17 aa, Synthetic construct
AAKSRLGWTLSPSAYNY

SEQ ID NO: 20 - protein, 6 aa, Synthetic construct
GDTANC

SEQ ID NO: 21 - protein, 8 aa, Synthetic construct
IYTGLGNT

SEQ ID NO: 22 - protein, 13 aa, Synthetic construct
AAGGILGRFCSRY

SEQ ID NO: 23 - protein, 8 aa, Synthetic construct
GFPYSTSR

SEQ ID NO: 24 - protein, 8 aa, Synthetic construct
IVPGTGYT

SEQ ID NO: 25 - protein, 19 aa, Synthetic construct
AAGDRRLLLNLLAPADYFY

SEQ ID NO: 26 - protein, 5 aa, Synthetic construct
IAYMA

SEQ ID NO: 27 - protein, 16 aa, Synthetic construct
LIATSGTTSYANSVKG

SEQ ID NO: 28 - protein, 16 aa, Synthetic construct
KSRLGWPGLETSQFKY

SEQ ID NO: 29 - skipped

SEQ ID NO: 30 - protein, 17 aa, Synthetic construct
AIVTQSGLTGYADSVKG

SEQ ID NO: 31 - protein, 15 aa, Synthetic construct
KSRLGWTLSPSAYNY

SEQ ID NO: 32 - skipped

SEQ ID NO: 33 - protein, 17 aa, Synthetic construct
AIYTGLGNTYYANSAKG

SEQ ID NO: 34 - protein, 11 aa, Synthetic construct
GGILGRFCSRY

SEQ ID NO: 35 - protein, 5 aa, Synthetic construct
TSRVG

SEQ ID NO: 36 - protein, 17 aa, Synthetic construct
TIVPGTGYTYYADSVKG

SEQ ID NO: 37 - protein, 17 aa, Synthetic construct
GDRRLLLNLLAPADYFY

SEQ ID NO: 38 - protein, 7 aa, Synthetic construct
GYAGGIA

SEQ ID NO: 39 - protein, 5 aa, Synthetic construct
ATSGT

SEQ ID NO: 40 - skipped

SEQ ID NO: 41 - protein, 6 aa, Synthetic construct
VTQSGL

SEQ ID NO: 42 - protein, 5 aa, Synthetic construct
GDTAN

SEQ ID NO: 43 - protein, 6 aa, Synthetic construct
YTGLGN

SEQ ID NO: 44 - protein, 7 aa, Synthetic construct
GFPYSTS

SEQ ID NO: 45 - protein, 6 aa, Synthetic construct
VPGTGY

SEQ ID NO: 46 - protein, 98 aa, Synthetic construct
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCAR

SEQ ID NO: 47 - protein, 98 aa, Synthetic construct
QVQLVESGGGVVQPGRSLRLSCAASGFTFSSYGMHWVRQAPGKGLEWVAVISYDGSNKYYADSVKGRFTISRDNSKNRLYLQMNSLRAEDTAVYYCAR

SEQ ID NO: 48 - protein, 11 aa, Synthetic construct
WGQGTMVTVSS

SEQ ID NO: 49 - protein, 11 aa, Synthetic construct
WGRGTLVTVSS

SEQ ID NO: 50 - protein, 124 aa, Synthetic construct
EVQLVESGGGLVQPGGSLRLSCAASGYAGGIAYMAWFRQAPGKGREGVANIATSGTTYYVDSVKGRFTISRDGAKNSLYLQMNSLRAEDTAVYYCAAKSRLGWPGLETSQFKYWGQGTMVTVSS

SEQ ID NO: 51 - protein, 124 aa, Synthetic construct
EVQLVESGGGLVQPGGSLRLSCAASGYAGGIAYMAWFRQAPGKGREGVALIATSGTTSYVDSVKGRFTISRDGAKNSLYLQMNSLRAEDTAVYYCAAKSRLGWPGLETSQFKYWGQGTMVTVSS

SEQ ID NO: 52 - protein, 124 aa, Synthetic construct
EVQLVASGGGLVQPGGSLRLSCAASGYAGGIAYMAWFRQAPGKGREGVALIATSGTTSYVDSVKGRFTISRDGAKNSVYLQMNSLRAEDTAVYYCAAKSRLGWPGLETSQFKYWGQGTMVTVSS

SEQ ID NO: 53 - protein, 124 aa, Synthetic construct
EVQLVESGGGLVQPGGSLRLSCAASGYAGGIAYMAWFRQAPGKGREGVALIATSGTTSYADSVKGRFTISRDGAKNSVYLQMNSLRAEDTAVYYCAAKSRLGWPGLETSQFKYWGQGTMVTVSS

SEQ ID NO: 54 - protein, 124 aa, Synthetic construct
EVQLVESGGGLVQPGGSLRLSCAASGYAGGIAYMAWFRQAPGKGREGVALIATSGTTSYVDSVKGRFTISRDGADNSVYLQMNSLRAEDTAVYYCAAKSRLGWPGLETSQFKYWGQGTMVTVSS

SEQ ID NO: 55 - protein, 120 aa, Synthetic construct
EVQLVESGGGVVQPGRSLRLSCAASGYLYMGWFRQAPGKEREGVAVIVTQSGLTYYADSVKGRFTISRDNSKNRLYLQMNSLRAEDTAVYYCAAKSRLGWTLSPSAYNYWGRGTLVTVSS

SEQ ID NO: 56 - protein, 120 aa, Synthetic construct
EVQLVESGGGVVQPGRSLRLSCAASGYLYMGWFRQAPGKEREGVAVIVTQSGLTYYADSVKGRFTISQDNSKNRLYLQMNSLRAEDTAVYYCAAKSRLGWTLSPSAYNYWGRGTLVTVSS

SEQ ID NO: 57 - protein, 120 aa, Synthetic construct
EVQLVESGGGVVQPGRSLRLSCAASGYLYMGWFRQAPGKEREGVAAIVTQSGLTYYADSVKGRFTISQDNSKNRLYLQMNSLRAEDTAVYYCAAKSRLGWTLSPSAYNYWGRGTLVTVSS

SEQ ID NO: 58 - protein, 120 aa, Synthetic construct
EVQLVESGGGVVQPGRSLRLSCAASGYLYMGWFRQAPGKEREGVAAIVTQSGLTGYADSVKGRFTISQDNSKNRLYLQMNSLRAEDTAVYYCAAKSRLGWTLSPSAYNYWGRGTLVTVSS

SEQ ID NO: 59 - protein, 120 aa, Synthetic construct
EVQLVESGGGVVQPGRSLRLSCAASGYLYMGWFRQAPGKEREGVAAIVTQSGLTGYADSVKGRFTISQDNAKNRLYLQMNSLRAEDTAVYYCAAKSRLGWTLSPSAYNYWGRGTLVTVSS

SEQ ID NO: 60 - protein, 120 aa, Synthetic construct
DVQLVESGGGVVQPGRSLRLSCAASGYLYMGWFRQAPGKEREGVAAIVTQSGLTGYADSVKGRFTISQDNSKNRLYLQMNSLRAEDTAVYYCAAKSRLGWTLSPSAYNYWGRGTLVTVSS

SEQ ID NO: 61 - protein, 120 aa, Synthetic construct
EVQLQASGGGVVQPGRSLRLSCAASGYLYMGWFRQAPGKEREGVAAIVTQSGLTGYADSVKGRFTISQDNSKNRLYLQMNSLRAEDTAVYYCAAKSRLGWTLSPSAYNYWGRGTLVTVSS

SEQ ID NO: 62 - protein, 120 aa, Synthetic construct
EVQLVESGGGVVQPGRSLRLSCAASGYLYMGWFRQAPGKEREGVAAIVTQSGLTGYADSVKGRFTISQDNSKNRIYLQMNSLRAEDTAVYYCAAKSRLGWTLSPSAYNYWGRGTLVTVSS

SEQ ID NO: 63 - protein, 118 aa, Synthetic construct
EVQLVESGGGLVQPGGSLRLSCAASGDTANCIGWFRQAPGKGREGVAAIYTGLGNTYYANSAKGRFTISQDNAKNSVYLQMNSLRAEDTAVYYCAAGGILGRFCSRYFGQGTMVTVSS

SEQ ID NO: 64 - protein, 118 aa, Synthetic construct
EVQLVESGGGLVQPGGSLRLSCAASGDTANCIGWFRQAPGKGLEGVAAIYTGLGNTYYANSAKGRFTISQDNAKNSVYLQMNSLRAEDTAVYYCAAGGILGRFCSRYFGQGTMVTVSS

SEQ ID NO: 65 - protein, 118 aa, Synthetic construct
EVQLVESGGGLVQPGGSLRLSCAASGDTASCIGWFRQAPGKGREGVAAIYTGLGNTYYANSAKGRFTISQDNAKNSVYLQMNSLRAEDTAVYYCAAGGILGRFCSRYFGQGTMVTVSS

SEQ ID NO: 66 - protein, 118 aa, Synthetic construct
EVQLVESGGGLVQPGGSLRLSCAASGDTANCIGWFRQAPGKGREGVAAIYTGLGNTYYANSAKGRFTISRDNAKNSVYLQMNSLRAEDTAVYYCAAGGILGRFCSRYFGQGTMVTVSS

SEQ ID NO: 67 - protein, 118 aa, Synthetic construct
DVQLVESGGGLVQPGGSLRLSCAASGDTANCIGWFRQAPGKGREGVAAIYTGLGNTYYANSAKGRFTISQDNAKNSVYLQMNSLRAEDTAVYYCAAGGILGRFCSRYFGQGTMVTVSS

SEQ ID NO: 68 - protein, 118 aa, Synthetic construct
EVQLVASGGGLVQPGGSLRLSCAASGDTANCIGWFRQAPGKGREGVAAIYTGLGNTYYANSAKGRFTISQDNAKNSVYLQMNSLRAEDTAVYYCAAGGILGRFCSRYFGQGTMVTVSS

SEQ ID NO: 69 - protein, 126 aa, Synthetic construct
EVQLVESGGGLVQPGGSLRLSCAASGFPYSTSRMGWFRQAPGKEREGVATIVPGTGYTYYVDSVKGRFTISQDNAKNSLYLQMNSLRAEDTAVYYCAAGDRRLLLNLLAPADYFYWGQGTMVTVSS

SEQ ID NO: 70 - protein, 126 aa, Synthetic construct
EVQLVESGGGLVQPGGSLRLSCAASGFPYSTSRMGWFRQAPGKEREGVATIVPGTGYTYYVDSVKGRFTISQDSAKNSLYLQMNSLRAEDTAVYYCAAGDRRLLLNLLAPADYFYWGQGTMVTVSS

SEQ ID NO: 71 - protein, 126 aa, Synthetic construct
EVQLVESGGGLVQPGGSLRLSCAASGFPYSTSRVGWFRQAPGKEREGVATIVPGTGYTYYVDSVKGRFTISQDNAKNSVYLQMNSLRAEDTAVYYCAAGDRRLLLNLLAPADYFYWGQGTMVTVSS

SEQ ID NO: 72 - protein, 126 aa, Synthetic construct
EVQLVASGGGLVQPGGSLRLSCAASGFPYSTSRMGWFRQAPGKEREGVATIVPGTGYTYYVDSVKGRFTISQDNAKNSLYLQMNSLRAEDTAVYYCAAGDRRLLLNLLAPADYFYWGQGTMVTVSS

SEQ ID NO: 73 - protein, 126 aa, Synthetic construct
EVQLQESGGGLVQPGGSLRLSCVASGFPYSTSRMGWFRQAPGKEREGVATIVPGTGYTYYVDSVKGRFTISQDNAKNSLYLQMNSLRAEDTAVYYCAAGDRRLLLNLLAPADYFYWGQGTMVTVSS

SEQ ID NO: 74 - protein, 126 aa, Synthetic construct
EVQLVASGGGLVQPGGSLRLSCAASGFPYSTSRMGWFRQAPGKEREGVATIVPGTGYTYYVDSVKGRFTISQDSAKNSLYLQMNSLRAEDTAVYYCAAGDRRLLLNLLAPADYFYWGQGTMVTVSS

SEQ ID NO: 75 - protein, 126 aa, Synthetic construct
EVQLVASGGGLVQPGGSLRLSCAASGFPYSTSRMGWFRQAPGKEREGVATIVPGTGYTYYADSVKGRFTISQDNAKNSLYLQMNSLRAEDTAVYYCAAGDRRLLLNLLAPADYFYWGQGTMVTVSS

SEQ ID NO: 76 - protein, 114 aa, Synthetic construct
QVQLVESGGGLVQPGGSLRLSCAASGILVSAYVMAWYRQPPGKQRDLVARLSTDGRTTYADSVKGRFTISKDNAKSTLYLQMNTLKSEDTAVYYCYAERSRGHWGQGTQVTVSS

SEQ ID NO: 77 - protein, 115 aa, Synthetic construct
QVQLVESGGGLVQPGGSLRLSCTASGFTFSSAWMYWFRQAPGKGLEWVSHIDPRGGSTYYADSVKGRFTISRDNAKNTLYLQMRSLKSEDTAVYYCAKDLRGLGRGQGTQVTVSS

SEQ ID NO: 78 - protein, 8 aa, Synthetic construct
GILVSAYV

SEQ ID NO: 79 - protein, 7 aa, Synthetic construct
LSTDGRT

SEQ ID NO: 80 - protein, 8 aa, Synthetic construct
YAERSRGH

SEQ ID NO: 81 - protein, 8 aa, Synthetic construct
GFTFSSAW

SEQ ID NO: 82 - protein, 8 aa, Synthetic construct
IDPRGGST

SEQ ID NO: 83 - protein, 8 aa, Synthetic construct
AKDLRGLG

SEQ ID NO: 84 - protein, 5 aa, Synthetic construct
AYVMA

SEQ ID NO: 85 - protein, 16 aa, Synthetic construct
RLSTDGRTTYADSVKG

SEQ ID NO: 86 - protein, 6 aa, Synthetic construct
ERSRGH

SEQ ID NO: 87 - protein, 5 aa, Synthetic construct
SAWMY

SEQ ID NO: 88 - protein, 17 aa, Synthetic construct
HIDPRGGSTYYADSVKG

SEQ ID NO: 89 - protein, 6 aa, Synthetic construct
DLRGLG

SEQ ID NO: 90 - protein, 7 aa, Synthetic construct
GILVSAY

SEQ ID NO: 91 - protein, 5 aa, Synthetic construct
STDGR

SEQ ID NO: 92 - protein, 7 aa, Synthetic construct
GFTFSSA

SEQ ID NO: 93 - protein, 6 aa, Synthetic construct
DPRGGS

SEQ ID NO: 94 - protein, 98 aa, Synthetic construct
EVQLVESGGGLVQPGGSLRLSCAASGFTFSSYWMHWVRQAPGKGLVWVSRINSDGSSTTYADSVKGRFTISRDNAKNTLYLQMNSLRAEDTAVYYCAR

SEQ ID NO: 95 - protein, 98 aa, Synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 96 - protein, 114 aa, Synthetic construct
EVQLVESGGGLVQPGGSLRLSCAASGILVSAYVMAWYRQAPGKQRVLVSRLSTDGRTTYADSVKGRFTISKDNAKNTLYLQMNSLRAEDTAVYYCYAERSRGHWGQGTMVTVSS

SEQ ID NO: 97 - protein, 114 aa, Synthetic construct
EVQLVESGGGLVQPGGSLRLSCAASGILVSAYVMAWYRQAPGKQRVLVSRLSTDGRTTYADSVKGRFTISKDNAKSTLYLQMNSLRAEDTAVYYCYAERSRGHWGQGTMVTVSS

SEQ ID NO: 98 - protein, 115 aa, Synthetic construct
EVQLVESGGGLVQPGGSLRLSCAASGFTFSSAWMYWFRQAPGKGLEWVSHIDPRGGSTYYADSVKGRFTISRDNSKNTLYLQMNSLRAEDTAVYYCAKDLRGLGRGQGTMVTVSS

SEQ ID NO: 99 - protein, 115 aa, Synthetic construct
EVQLVESGGGLVQPGGSLRLSCAASGFTFSSAWMYWFRQAPGKGLEWVSHIDPRGGSTYYADSVKGRFTISRDNAKNTLYLQMNSLRAEDTAVYYCAKDLRGLGRGQGTQVTVSS

SEQ ID NO: 100 - protein, 119 aa, Synthetic construct
QVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSS

SEQ ID NO: 101 - protein, 109 aa, Synthetic construct
SSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVL

(19) United States
(12) Patent Application Publication
(10) Pub. No.: US 2026/0285971 A1
(43) Pub. Date: Sep. 24, 2026

(54) ANTI-GPRC5D NANOBODY AND USE THEREOF

(71) Applicant: Shandong Simcere Biopharmaceutical Co., Ltd., Yantai, Shandong (CN)

(72) Inventors: Guangcun Cheng, Shanghai (CN); Yayuan Fu, Shanghai (CN); Ling Li, Shanghai (CN); Yifang Wang, Shanghai (CN); Zhuoxiao Cao, Shanghai (CN); Renhong Tang, Shanghai (CN)

(21) Appl. No.: 18/996,106

(22) PCT Filed: Jul. 20, 2023
(86) PCT No.: PCT/CN2023/108360

§ 371 Date: Jan. 17, 2025

(30) Foreign Application Priority Data
Jul. 21, 2022 (CN) 202210860364.1

(51) Int. Cl. C07K 16/28; A61K 47/68; G01N 33/575

(52) U.S. Cl. CPC C07K 16/28; A61K 47/6849; G01N 33/5759; C07K 2317/24; C07K 2317/31; C07K 2317/565; C07K 2317/569; C07K 2317/92; G01N 2333/726

(57) ABSTRACT

Provided in the present disclosure are an antibody against G-protein-coupled receptor C5 family subtype D (GPRC5D), and the use thereof. Specifically, disclosed in the present application are a nanobody or an antigen-binding fragment thereof that specifically binds to GPRC5D, a nucleic acid encoding same, an expression vector and an expression cell thereof, a preparation method therefor, a pharmaceutical composition thereof, and the use thereof in the preparation of a pharmaceutical composition for treating diseases, for example, the use thereof in treating tumors. Compared with full-length antibodies, the nanobody provided in the present disclosure can bind more effectively to the target protein GPRC5D against which it is difficult to develop an antibody. The present disclosure has great significance for the development of a therapeutic drug and detection reagent for the GPRC5D antibody.

### CROSS-REFERENCE TO RELATED PATENT APPLICATION

The present application claims priority to the Chinese Patent Application No. 202210860364.1 filed with China National Intellectual Property Administration on Jul. 21, 2022, and entitled “ANTI-GPRC5D NANOBODY AND USE THEREOF”, which is incorporated herein by reference in its entirety.

### TECHNICAL FIELD

The present application relates to the field of antibodies, and in particular to an anti-GPRC5D nanobody.

### BACKGROUND

A special antibody lacking a light chain exists in animals of the Camelidae family and cartilaginous fish. A variable region fragment derived from this antibody is generally called a nanobody, which has a molecular weight of only 15 kD. A nanobody generally has a long CDR3, can form a stable large convex ring structure, can bind to some hidden epitopes, and thus is particularly suitable for targets where antibodies are difficult to obtain, such as GPCRs, ion channels, and enzyme activity centers. Compared with traditional antibodies, the VHH nanobody features small molecular weight, easy in vitro expression, good solubility, and weak immunogenicity, and can penetrate through some protective barriers in a body, e.g., the blood-brain barrier, to enter the diseased region to play a role.

Multiple myeloma (MM) is a plasma cell malignant tumor characterized by the unrestricted proliferation of plasma cells in the bone marrow like tumor cells, accompanied by secretion of monoclonal immunoglobulins, which causes a series of clinical manifestations such as multiple osteolytic damage, hypercalcemia, anemia, kidney damage, and repeated infections. Combined treatment with hematopoietic stem cell transplantation and immunomodulators and protease inhibitors can significantly improve the prognosis. In recent years, a breakthrough has been made in CAR T therapy for the treatment of multiple myeloma. In particular, CAR T targeting B cell maturation antigen (BCMA) has shown positive clinical results in the treatment of multiple myeloma. However, multiple myeloma patients who are BCMA negative or have low BCMA expression would still relapse after receiving BCMA-targeted CAR-T cell therapy. Therefore, the treatment of multiple myeloma still requires the identification of other new targets with greater potential.

GPRC5D belongs to the G protein-coupled receptors (GPCRs) and is an orphan receptor. It is the G protein-coupled receptor class C group 5 member D and is a seven-transmembrane protein. GPRC5D has three exons. The amino-terminal domain is short, and its ligand and signaling mechanisms are not well defined. GPRC5D is highly conserved across different species, with human GPRC5D having 92% homology to cynomolgus monkey GPRC5D. GPRC5D is highly expressed in multiple myeloma cells, but it is hardly expressed in normal tissues, with the expression limited to the hair follicle region. Because hair follicles are immune-privileged sites, GPRC5D is an ideal target for the treatment of multiple myeloma (MM) based on this feature. GPRC5D is mainly expressed in malignant plasma cells of MM patients and has better specificity compared with BCMA. Meanwhile, since GPRC5D and BCMA are independently expressed, either a single targeting therapeutic drug or a dual targeting therapeutic drug can be developed. Currently, in the field of multiple myeloma treatment, there is extensive research on BCMA-targeted immunotherapies. However, there is a lack of effective treatment options for MM patients who are BCMA-negative or have low BCMA expression, as well as for those who experience disease relapse due to antigen immune escape following BCMA-targeted therapy. Therefore, the development of therapeutic drugs targeting GPRC5D is expected to be the key to solving this problem.

### SUMMARY

The present disclosure provides a nanobody capable of specifically binding to G protein-coupled receptor class C group 5 member D (GPRC5D). Compared with full-length antibodies, the nanobody is capable of better binding to such antigens as GPRC5D that are difficult to bind to. Meanwhile, a novel treatment means is provided for patients with no expression or low expression of BCMA and with poor prognosis for BCMA-targeted treatment.

The present disclosure further provides an antibody or an antigen-binding fragment thereof that specifically binds to GPRC5D, a multispecific antigen-binding molecule, a nucleic acid fragment, a vector, a host cell, a preparation method, a pharmaceutical composition, pharmaceutical use, and a method for treating a tumor or cancer (e.g., B cell lymphoma or multiple myeloma).

In a first aspect, the present disclosure provides an antibody or an antigen-binding fragment thereof that specifically binds to human or monkey GPRC5D, which comprises a CDR1, a CDR2, and a CDR3, wherein the CDR1, the CDR2, and the CDR3 comprise an HCDR1, an HCDR2, and an HCDR3, respectively, of a VHH domain set forth in any one of SEQ ID NOs: 10-13, 50-75, 76-77, and 96-99.

In a second aspect, the present disclosure further provides a multispecific antigen-binding molecule, which comprises the antibody or the antigen-binding fragment thereof described above and an antigen-binding molecule binding to an antigen other than GPRC5D or binding to a GPRC5D epitope different from that of the antibody or the antigen-binding fragment thereof described above.

In a third aspect, the present disclosure further provides an isolated nucleic acid fragment encoding the antibody or the antigen-binding fragment thereof described above or the multispecific antigen-binding molecule described above.

In a fourth aspect, the present disclosure further provides a vector, which comprises the nucleic acid fragment described above.

In a fifth aspect, the present disclosure further provides a host cell, which comprises the nucleic acid fragment described above or the vector described above.

In a sixth aspect, the present disclosure further provides a method for preparing the antibody or the antigen-binding fragment thereof described above or the multispecific antigen-binding molecule described above, which comprises culturing the host cell described above, and isolating an antibody or an antigen-binding fragment thereof or a multispecific antigen-binding molecule expressed by the host cell.

In a seventh aspect, the present disclosure further provides a pharmaceutical composition, which comprises the antibody or the antigen-binding fragment thereof described above, the multispecific antigen-binding molecule described above, the nucleic acid fragment described above, the vector described above, or a product prepared by the method described above.

In an eighth aspect, the present disclosure further provides a method for treating a tumor or cancer, which comprises administering to a subject an effective amount of the antibody or the antigen-binding fragment thereof described above, the multispecific antigen-binding molecule described above, the nucleic acid fragment described above, the vector described above, a product prepared by the method described above, or the pharmaceutical composition described above.

In a ninth aspect, the present disclosure further provides use of the antibody or the antigen-binding fragment thereof described above, the multispecific antigen-binding molecule described above, the nucleic acid fragment described above, the vector described above, a product prepared by the method described above, or the pharmaceutical composition described above in preparing a medicament for treating a tumor or cancer.

In a tenth aspect, the present disclosure further provides the antibody or the antigen-binding fragment thereof described above, the multispecific antigen-binding molecule described above, the nucleic acid fragment described above, the vector described above, a product prepared by the method described above, or the pharmaceutical composition described above for use in treating a tumor or cancer.

In an eleventh aspect, the present disclosure further provides a kit, which comprises the antibody or the antigen-binding fragment thereof described above, the multispecific antigen-binding molecule described above, the nucleic acid fragment described above, the vector described above, a product prepared by the method described above, or the pharmaceutical composition described above.

In a twelfth aspect, the present disclosure further provides a method for detecting expression of GPRC5D in a biological sample, which comprises contacting the biological sample with the antibody or the antigen-binding fragment thereof described above under conditions that allow the antibody or the antigen-binding fragment thereof and GPRC5D to form a complex.

In a thirteenth aspect, the present disclosure further provides use of the antibody or the antigen-binding fragment thereof described above in preparing a GPRC5D detection reagent.

Beneficial effects: compared with a full-length antibody, the antibody provided by the present disclosure has excellent binding capacity for human and monkey GPRC5D antigens, and the development of drugs and immunotherapies based on the antibody of the present disclosure has high application value for improving the survival rate and prognosis of myeloma patients experiencing relapse following BCMA-targeted therapy or myeloma patients who are BCMA-negative.

### BRIEF DESCRIPTION OF THE DRAWINGS

FIGS. 1A-1C show the expression levels in 293T-human GPRC5D, CHO-K1-human GPRC5D, and 293T-cyno GPRC5D cell lines assayed by FACS.

FIG. 2 shows the binding reactions of Lab01-huFc, Lab02-huFc, Lab03-huFc, and Lab04-huFc recombinant antibodies with human multiple myeloma cells NCI-H929 assayed by FACS.

FIG. 3A shows the binding reactions of Lab01-huFc, Lab02-buFc, Lab03-huFc, and Lab04-huFc recombinant antibodies with the overexpression cell 239T-human GPRC5D assayed by FACS.

FIG. 3B shows the binding reactions of Lab01-huFc, Lab02-huFc, Lab03-huFc, and Lab04-huFc recombinant antibodies with the overexpression cell 239T-cyno GPRC5D assayed by FACS.

FIG. 3C shows the binding reactions of Lab01-huFc, Lab02-huFc, Lab03-huFc, and Lab04-huFc recombinant antibodies with the negative control cell 239T assayed by FACS.

FIGS. 4A-4D show the binding reactions of HAB01, HAB02, HAB03, and HAB04 humanized antibodies with human GPRC5D protein, respectively, assayed by ELISA.

FIGS. 5A-5D show the binding reactions of HAB01, HAB02, HAB03, and HAB04 humanized antibodies with human multiple myeloma cell NCI-H929, respectively, assayed by FACS.

FIGS. 6A-6D show the binding reactions of HAB01, HAB02, HAB03, and HAB04 humanized antibodies with human multiple myeloma cell molp-8, respectively, assayed by FACS.

FIGS. 7A-7D show the binding reactions of HAB01, HAB02, HAB03, and HAB04 humanized antibodies with human multiple myeloma cell RPMI-8226, respectively, assayed by FACS.

FIG. 8 shows the binding reactions of recombinant antibodies and humanized antibodies with overexpression cell CHOK1-human GPRC5D assayed by FACS.

FIG. 9 shows the binding reactions of the recombinant antibodies and the humanized antibodies with human multiple myeloma cell NCI-H929 assayed by FACS.

FIG. 10 shows the binding reactions of the recombinant antibodies and the humanized antibodies with human multiple myeloma cell molp-8 assayed by FACS.

FIG. 11 shows the binding reactions of the recombinant antibodies and the humanized antibodies with human multiple myeloma cell RPMI-8226 assayed by FACS.

### TERMINOLOGY AND DEFINITIONS

Unless otherwise defined herein, scientific and technical terms used in correlation with the present disclosure shall have the meanings that are commonly understood by those skilled in the art. Furthermore, unless otherwise stated herein, terms used in the singular form herein shall include the plural form, and vice versa. More specifically, as used in this specification and the appended claims, unless otherwise clearly indicated, the singular forms “a”, “an”, and “the” include referents in the plural form.

The terms “include”, “comprise”, and “have” herein are used interchangeably and are intended to indicate the inclusion of a solution, implying that there may be elements other than those listed in the solution. Meanwhile, it should be understood that the descriptions “include”, “comprise”, and “have” used herein, in addition to the above meaning, also represent the solution of “consist of . . . ”. Illustratively, “a composition, comprising A and B” should be understood as the following technical solution: a composition consisting of A and B, and a composition containing other components in addition to A and B, all fall within the scope of the aforementioned “a composition”.

The term “and/or” used herein includes the meanings of “and”, “or”, and “all or any other combination of elements linked by the term”.

The term GPRC5D herein refers to the G protein-coupled receptor class C group 5 member D. It is an orphan receptor, and it is a seven-transmembrane protein and currently has no known ligand. The GPRC5D is highly expressed on the surface of primary multiple myeloma cells, while its expression in normal tissues is limited to the hair follicle region. Studies have shown that 65% of patients with multiple myeloma exceed the GPRC5D expression threshold of 50%, and by virtue of this characteristic, GPRC5D is a potential target for treating MM.

The term “specifically bind to” herein refers to that an antigen-binding molecule (e.g., an antibody) specifically binds to an antigen and substantially identical antigens, generally with high affinity, but does not bind to unrelated antigens with high affinity. The affinity is generally reflected in an equilibrium dissociation constant (KD), where a relatively low KD indicates a relatively high affinity. In the case of antibodies, high affinity generally means having a KD of about 10⁻⁶ M or less, 10⁻⁷ M or less, about 10⁻⁸ M or less, or about 10⁻⁹ M or less. The KD is calculated as follows: KD=Kd/Ka, where Kd represents the dissociation rate and Ka represents the association rate. The equilibrium dissociation constant KD can be measured by methods well known in the art, such as surface plasmon resonance (e.g., Biacore) or equilibrium dialysis.

The term “antigen-binding molecule” herein is used in its broadest sense and refers to a molecule that specifically binds to an antigen. Illustratively, the antigen-binding molecule includes, but is not limited to, an antibody or an antibody mimetic. “Antibody mimetic” refers to an organic compound or a binding domain that is capable of specifically binding to an antigen, but is not structurally related to an antibody. Illustratively, the antibody mimetic includes, but is not limited to, affibody, affitin, affilin, a designed ankyrin repeat protein (DARPin), a nucleic acid aptamer, and a Kunitz domain peptide.

The term “antibody” herein is used in its broadest sense and refers to a polypeptide or a combination of polypeptides that comprises sufficient sequence from an immunoglobulin heavy chain variable region and/or sufficient sequence from an immunoglobulin light chain variable region to be capable of specifically binding to an antigen. “Antibody” herein encompasses various forms and various structures as long as they exhibit the desired antigen binding activity. “Antibody” herein includes alternative protein scaffolds or artificial scaffolds having grafted complementarity determining regions (CDRs) or CDR derivatives. Such scaffolds include antibody-derived scaffolds comprising mutations introduced to, for example, stabilize the three-dimensional structure of the antibody, and fully synthetic scaffolds comprising, for example, biocompatible polymers. Such scaffolds may also include non-antibody-derived scaffolds, such as scaffold proteins known in the art to be useful for grafting CDRs, including but not limited to, tenascin, fibronectin, peptide aptamers, and the like.

The term “antibody” includes intact antibodies and any antigen-binding fragments (i.e., “antigen-binding moiety”) or single chains thereof. The “antibody” includes a glycoprotein or an antigen-binding moiety thereof having at least two heavy (H) chains and two light (L) chains that are linked together by disulfide bonds. Each heavy chain consists of a heavy chain variable region (abbreviated herein as VH) and a heavy chain constant region. The heavy chain constant region consists of three domains: CH1, CH2, and CH3. Each light chain consists of a light chain variable region (abbreviated herein as VL) and a light chain constant region. The light chain constant region consists of one domain CL. The VH region and the VL region can be further subdivided into hypervariable regions called complementarity determining regions (CDRs), which are scattered among regions that are more conserved, termed framework regions (FRs). Each VH or VL consists of three CDRs and four FRs, which are arranged from the N-terminus to the C-terminus in the following order: FR1, CDR1, FR2, CDR2, FR3, CDR3, and FR4. The variable regions of the heavy and light chains comprise binding domains that can interact with antigens. The constant regions of the antibody may mediate the binding of the immunoglobulin to a host tissue or factor, and the host tissue or factor includes various cells (e.g., effector cells) of the immune system and the first component (C1q) of the classical complement system. The heavy chain constant regions of immunoglobulins differ in their amino acid composition and arrangement, and thus in their antigenicity. Accordingly, “immunoglobulin” herein can be divided into five classes, or isoforms of immunoglobulins, i.e., IgM, IgD, IgG, IgA, and IgE, with their corresponding heavy chains being ρ, δ, γ, α, and ε chains, respectively. The Ig of the same class may also be divided into different subclasses according to the differences in the amino acid composition of the hinge regions and the number and location of disulfide bonds in the heavy chains. For example, IgG may be divided into IgG1, IgG2, IgG3, and IgG4, and IgA may be divided into IgA1 and IgA2. The light chains can be divided into κ or λ chains according to the differences in the constant regions. Each of the five classes of Ig may have a κ chain or λ chain.

The “antibody” herein also includes antibodies that do not comprise a light chain, e.g., heavy chain antibodies (HCAbs) produced by Camelidae species, e.g., Camelus dromedarius, Camelus bactrianus, Lama glama, Lama guanicoe, Vicugna pacos, and the like, as well as immunoglobulin new antigen receptors (IgNARs) found in Chondrichthyes, e.g., shark.

The term “antibody” herein may be derived from any animal, including but not limited to human and non-human animals which may be selected from primates, mammals, rodents, and vertebrates, such as Camelidae species, Lama glama, Lama guanicoe, Vicugna pacos, sheep, rabbits, mice, rats, or Chondrichthyes (e.g., shark).

The term “heavy chain antibody” herein refers to an antibody lacking a light chain of a conventional antibody. The term specifically includes, but is not limited to, homodimeric antibodies comprising a VH antigen-binding domain and CH2 and CH3 constant domains in the absence of a CH1 domain.

The term “nanobody” herein refers to a heavy chain antibody naturally lacking a light chain present in a camel or the like, and the cloning of its variable region can give a single domain antibody only consisting of a heavy chain variable region (also called VHH (variable domain of heavy chain of heavy chain antibody)), which is the smallest functional antigen-binding fragment.

The terms “VHH domain”, “nanobody”, and “single domain antibody” (sdAb) herein have the same meaning and are used interchangeably, and refer to a single domain antibody consisting of only one heavy chain variable region constructed by cloning a variable region of a heavy chain antibody, which is the smallest antigen-binding fragment having the complete functionality. Generally, a single domain antibody consisting of only one heavy chain variable region is constructed by acquiring a heavy chain antibody naturally lacking a light chain and a heavy chain constant region 1 (CH1) and then cloning the variable region of the antibody heavy chain.

For further description of “heavy chain antibodies”, “single domain antibodies”, “VHH domains”, and “nanobodies”, see the following patent applications mentioned as general background art: WO 94/04678, WO 95/04079 and WO 96/34103; WO 94/25591, WO 99/37681, WO 00/40968, WO 00/43507, WO 00/65057, WO 01/40310, WO 01/44301, EP 1134231 and WO 02/48193; WO97/49805, WO 01/21817, WO 03/035694, WO 03/054016 and WO 03/055527; WO 03/050531; WO 01/90190; WO03/025020; and WO 04/041867, WO 04/041862, WO 04/041865, WO 04/041863, WO 04/062551, WO 05/044858, WO 06/40153, WO 06/079372, WO 06/122786, WO 06/122787 and WO 06/122825; and other prior art mentioned in these applications.

The term “multispecific” herein refers to the ability of an antibody or an antigen-binding fragment thereof to bind to, e.g., different antigens or to at least two different epitopes on the same antigen. Thus, the terms such as “bispecific”, “trispecific”, and “tetraspecific” refer to the number of different epitopes to which an antibody can bind. For example, conventional monospecific IgG antibodies have two identical antigen-binding sites (paratopes) and therefore can only bind to the same epitope (rather than to different epitopes). In contrast, multispecific antibodies have at least two different types of paratopes/binding sites and can therefore bind to at least two different epitopes. As used herein, the “complementarity determining region” refers to the antigen-binding site of an antibody. In addition, a single “specificity” may refer to one, two, three, or more identical complementarity determining regions in a single antibody (the actual number of complementarity determining regions/binding sites in a single antibody molecule is referred to as the “valency”). For example, a single native IgG antibody is monospecific and bivalent, since it has two identical paratopes. Accordingly, a multispecific antibody comprises at least two (different) complementarity determining regions/binding sites. Thus, the term “multispecific antibody” refers to an antibody having more than one paratope and the ability to bind to two or more different epitopes. The term “multispecific antibody” particularly includes bispecific antibodies as defined above, but typically also includes proteins, e.g., antibodies and scaffolds specifically binding to three or more different epitopes, i.e., antibodies having three or more paratopes/binding sites.

The term “valent” herein refers to the presence of a specified number of binding sites in an antibody/antigen-binding molecule. Thus, the terms “monovalent”, “divalent”, “tetravalent”, and “hexavalent” refer to the presence of one binding site, two binding sites, four binding sites, and six binding sites, respectively, in an antibody/antigen-binding molecule.

The “full-length antibody”, “complete antibody”, and “intact antibody” are used interchangeably herein and refer to an antibody having a structure substantially similar to that of a natural antibody.

“Antigen-binding fragment” and “antibody fragment” herein are used interchangeably and do not have the entire structure of an intact antibody, but comprise only a portion of the intact antibody or a variant of the portion that has the ability to bind to an antigen. Illustratively, the “antigen-binding fragment” or “antibody fragment” herein includes, but is not limited to, a Fab, an F(ab′) 2, a Fab′, a Fab′-SH, an Fd, an Fv, an scFv, a diabody, and a single domain antibody.

The term “chimeric antibody” herein refers to an antibody having a variable region sequence of an immunoglobulin derived from one organism (e.g., rat, mouse, rabbit, or Vicugna pacos) and a constant region of an immunoglobulin derived from a different organism (e.g., human). Methods for producing chimeric antibodies are known in the art. See, e.g., U.S. Pat. No. 5,807,715A (which is incorporated herein by reference).

The term “humanized antibody” herein refers to a genetically engineered non-human antibody that has an amino acid sequence modified to increase the homology to the sequence of a human antibody. Generally, all or part of the CDRs of a humanized antibody is derived from a non-human antibody (donor antibody), and all or part of the non-CDRs (e.g., variable region FRs and/or constant regions) is derived from a human immunoglobulin (receptor antibody). The humanized antibody generally retains or partially retains the desired properties of the donor antibody, including but not limited to, antigen specificity, affinity, reactivity, the ability to increase the activity of immune cells, the ability to enhance an immune response, or the like.

The term “fully human antibody” herein refers to an antibody having variable regions in which both the FRs and CDRs are derived from human germline immunoglobulin sequences. Furthermore, if the antibody comprises constant regions, the constant regions are also derived from human germline immunoglobulin sequences. The fully human antibody herein may include amino acid residues that are not encoded by human germline immunoglobulin sequences (e.g., mutations introduced by random or site-specific mutagenesis in vitro or by somatic mutations in vivo). However, “fully human antibody” herein does not include antibodies in which CDR sequences derived from the germline of another mammalian species (e.g., mouse) have been grafted onto human framework sequences.

The term “variable region” herein refers to a region of a heavy or light chain of an antibody involved in the binding of the antibody to an antigen. The “heavy chain variable region” is used interchangeably with “VH” and “HCVR”, and the “light chain variable region” is used interchangeably with “VL” and “LCVR”. The heavy and light chain variable domains of natural antibodies generally have similar structures, each domain containing four conserved framework regions (FRs) and three hypervariable regions (HVRs). A single VH or VL domain may be sufficient to provide antigen binding specificity.

The terms “complementarity determining region” and “CDR” are used interchangeably herein and generally refer to hypervariable regions (HVRs) found in both the light and heavy chain variable domains. The highly conserved portions of the variable domains are called framework regions (FRs). As known in the art, the amino acid positions representing the hypervariable regions of an antibody may vary according to the context and various definitions known in the art. Some positions within a variable domain may be considered to be hybrid hypervariable positions in that such positions may be considered to be within a hypervariable region according to one set of standards (such as IMGT or KABAT) but outside a hypervariable region according to a different set of standards (such as KABAT or IMGT). One or more of such positions may also be found in extended hypervariable regions. The present disclosure includes antibodies comprising modifications in such hybrid hypervariable positions. The heavy chain variable region CDRs may be abbreviated as HCDRs and the light chain variable region CDRs as LCDRs. The variable domains of native heavy and light chains each comprise four framework regions, mainly in a sheet configuration, connected by three CDRs (CDR1, CDR2, and CDR3) that form loops connecting the sheet structures, and in some cases form part of the sheet structures. The CDRs in each chain are held tightly together by the FR regions, in the order of FR1-CDR1-FR2-CDR2-FR3-CDR3-FR4, and contribute to the formation of the antigen-binding site of the antibody with CDRs from other antibody chains (see Kabat et al., Sequences of Proteins of Immunological Interest, National Institute of Health, Bethesda, Md. 1987, which is incorporated herein by reference).

The “CDR” herein may be annotated and defined in a manner well known in the art, including, but not limited to, Kabat numbering scheme, Chothia numbering scheme, or IMGT numbering scheme; the tool sites used include, but are not limited to, AbRSA site (http://cao.labshare.cn/AbRSA/cdrs.php), abYsis site (www.abysis.org/abysis/sequence_input/key_annotation/key_annotation.cgi), and IMGT site (http://www.imgt.org/3Dstructure-DB/cgi/DomainGapAlign.cgi #results). The CDR herein includes overlaps and subsets of amino acid residues defined in different ways.

The term “Kabat numbering scheme” herein generally refers to the immunoglobulin alignment and numbering scheme proposed by Elvin A. Kabat (see, e.g., Kabat et al., Sequences of Proteins of Immunological Interest, 5th Ed. Public Health Service, National Institutes of Health, Bethesda, Md., 1991).

The term “Chothia numbering scheme” herein generally refers to the immunoglobulin numbering scheme proposed by Chothia et al., which is a classical rule for identifying CDR region boundaries based on the position of structural loop regions (see, e.g., Chothia & Lesk (1987) J. Mol. Biol. 196:901-917; Chothia et al., (1989) Nature 342: 878-883).

The term “IMGT numbering scheme” herein generally refers to a numbering scheme based on the international ImMunoGeneTics information system (IMGT) initiated by Lefranc et al., see Lefranc et al., Dev. Comparat. Immunol. 27: 55-77, 2003.

The term “heavy chain constant region” herein refers to the carboxyl-terminal portion of an antibody heavy chain that is not directly involved in the binding of the antibody to an antigen, but exhibits effector functions, such as interaction with an Fc receptor; the heavy chain constant region has a more conserved amino acid sequence relative to the variable domain of the antibody. The “heavy chain constant region” may be selected from a CH1 domain, a hinge region, a CH2 domain, a CH3 domain, and a variant or fragment thereof. The “heavy chain constant region” includes a “full-length heavy chain constant region” having a structure substantially similar to that of a natural antibody constant region and a “heavy chain constant region fragment” including only “a portion of the full-length heavy chain constant region”. Illustratively, a typical “full-length antibody heavy chain constant region” consists of the CH1 domain-hinge region-CH2 domain-CH3 domain. When the antibody is IgE, it further comprises a CH4 domain; when the antibody is a heavy chain antibody, it does not comprise a CH1 domain. Illustratively, a typical “heavy chain constant region fragment” may be selected from an Fc domain and a CH3 domain.

The term “light chain constant region” herein refers to the carboxyl-terminal portion of an antibody light chain that is not directly involved in the binding of the antibody to an antigen. The light chain constant region may be selected from a constant κ domain and a constant λ domain.

The term “Fc region” herein is used to define the C-terminal region of an antibody heavy chain containing at least a portion of the constant region. The “Fc region” includes native Fc regions and variant Fc regions. Illustratively, the human IgG heavy chain Fc region may extend from Cys226 or Pro230 to the carboxyl-terminus of the heavy chain. However, an antibody produced by a host cell may undergo post-translational cleavage of one or more, particularly one or two, amino acids from the C-terminus of the heavy chain. Therefore, the antibody produced by a host cell via the expression of a specific nucleic acid molecule encoding a full-length heavy chain may include a full-length heavy chain, or may include cleaved variants of the full-length heavy chain. It may be such a situation when the last two C-terminal amino acids of the heavy chain are glycine (G446) and lysine (K447, in accordance with the Kabat EU index). Therefore, the C-terminal lysine (Lys447), or the C-terminal glycine (Gly446) and lysine (Lys447) of the Fc region, may or may not be present. Typically, the IgG Fc region comprises IgG CH2 and IgG CH3 domains; optionally, the IgG Fc region may further comprise a complete or partial hinge region, but does not comprise a CH1 domain. The “CH2 domain” of the human IgG Fc region typically extends from an amino acid residue at about position 231 to an amino acid residue at about position 340. In one embodiment, a carbohydrate chain is attached to the CH2 domain. The CH2 domain herein may be a CH2 domain of a native sequence or a variant CH2 domain. The “CH3 domain” comprises the residues in the Fc region at the C-terminus of the CH2 domain (i.e., from an amino acid residue at about position 341 to an amino acid residue at about position 447 of the IgG). The CH3 region herein may be a CH3 domain of a native sequence or a variant CH3 domain (e.g., a CH3 domain having a “protuberance” (“knob”) introduced in one strand and a “cavity” (“hole”) correspondingly introduced in the other strand; see U.S. Pat. No. 5,821,333, which is explicitly incorporated herein by reference). As described herein, such variant CH3 domains may be used to promote the heterodimerization of two non-identical antibody heavy chains.

Unless otherwise specified herein, amino acid residues in the Fc region or constant region are numbered according to the EU numbering scheme, also known as the EU index, as described in Kabat et al., Sequences of Proteins of Immunological Interest, 5^{th} Ed. Public Health Service, National Institutes of Health, Bethesda, MD, 1991.

The term “Fc variant” herein refers to a change in the structure or functionality of an Fe caused by the presence of one or more amino acid substitution, insertion, or deletion mutations at appropriate sites on the Fc. The “inter-Fc variant interaction” refers to the formation of space-filling effects, electrostatic steering, hydrogen bonding, hydrophobic interactions, etc., between mutationally designed Fc variants. The inter-Fc variant interaction contributes to the formation of stable heterodimeric proteins. Preferred mutational designs are those of the “knob-into-hole” format.

Techniques for designing mutations in Fc variants have been widely used in the art to produce bispecific antibodies or heterodimeric Fc fusion protein forms. Representatives are the “knob-into-hole” format proposed by Cater, et al.; the Fc-containing heterodimeric form produced by Amgen Inc. using electrostatic steering (US20100286374 A1); the heterodimeric form (SEEDbodies) formed by IgG/Ig chain exchange proposed by Jonathan H. Davis, et al.; the bispecific molecule produced by the DuoBody platform of Genmab A/S; the heterodimeric protein form given by Xencor by combining structural calculation and Fc amino acid mutation and different mechanisms of action (mAbs 3:6, 546-557; November/December 2011); the heterodimeric protein form produced by Alphamab Oncology, Suzhou, via the charge network-based Fc modification method (CN201110459100.7); and other genetic engineering methods to give heterodimeric functional proteins by means based on Fc amino acid change or functional modification. The knob/hole structures on the Fc variant fragment described herein refer to that the two Fc fragments are mutated separately and can be bound by the “knob-into-hole” format after mutation. The “knob-into-hole” model of Cater et al., is preferred for site-directed mutagenesis on the Fc region, such that the resulting first and second Fc variants can bind together in the “knob-into-hole” form to give a heterodimer. The selection of a particular immunoglobulin Fc region from a particular immunoglobulin class and subclass is within the knowledge of those skilled in the art. Preferred are the Fc regions of human IgG1, IgG2, IgG3, or IgG4 antibodies, and more preferred is the Fc region of human IgG1 antibody. One of the first Fc variant or the second Fc variant is randomly selected for the knob mutation and the other for hole mutation.

The term “conservative amino acid” herein generally refers to amino acids that belong to the same class or have similar characteristics (e.g., charge, side chain size, hydrophobicity, hydrophilicity, backbone conformation, and rigidity), Illustratively, the amino acids in each of the following groups belong to conservative amino acid residues of each other, and substitutions of amino acid residues within the groups belong to conservative amino acid substitutions:
1) alanine (A), serine(S), and threonine (T);
2) aspartic acid (D) and glutamic acid (E);
3) asparagine (N) and glutamine (Q);
4) arginine (R), lysine (K), and histidine (H);
5) isoleucine (I), leucine (L), methionine (M), and valine (V); and
6) phenylalanine (F), tyrosine (Y), and tryptophan (W).

The term “identity” herein can be acquired by a calculation as follows: to determine the percent “identity” of two amino acid sequences or two nucleic acid sequences, the sequences are aligned for optimal comparison purposes (e.g., for optimal alignment, gaps can be introduced in one or two of the first and second amino acid sequences or nucleic acid sequences, or non-homologous sequences can be discarded for comparison). Amino acid residues or nucleotides at corresponding amino acid positions or nucleotide positions are then compared. When a position in the first sequence is occupied by the same amino acid residue or nucleotide at the corresponding position in the second sequence, the molecules are identical at this position. The percent identity between two sequences varies with the identical positions shared by the sequences, taking into account the number of gaps that need to be introduced and the length of each gap for optimal alignment of the two sequences.

A mathematical algorithm can be used to compare two sequences and calculate the percent identity between the sequences. For example, the percent identity between two amino acid sequences is determined with the Needlema and Wunsch algorithm that has been integrated into the GAP program of the GCG software package (available at www.gcg.com), using the Blossum 62 matrix or PAM250 matrix and gap weight of 16, 14, 12, 10, 8, 6, or 4 and length weight of 1, 2, 3, 4, 5, or 6. For another example, the percent identity between two nucleotide sequences is determined with the GAP program of the GCG software package (available at www.gcg.com), using the NWSgapdna.CMP matrix and gap weight of 40, 50, 60, 70, or 80 and length weight of 1, 2, 3, 4, 5, or 6. A particularly preferred parameter set (and one that should be used unless otherwise stated) is a Blossum62 scoring matrix with a gap penalty of 12, a gap extension penalty of 4, and a frameshift gap penalty of 5. The percent identity between two amino acid sequences or nucleotide sequences can also be determined with a PAM120 weighted remainder table, a gap length penalty of 12, and a gap penalty of 4, using the E. Meyers and W. Miller algorithm that has been incorporated into the ALIGN program (version 2.0).

Additionally or alternatively, the nucleic acid sequences and protein sequences described herein can be further used as “query sequences” for searches against public databases to, e.g., identify sequences of other family members or correlated sequences. Such searches may be performed, for example, using NBLAST and XBLAST programs (version 2.0). BLAST nucleotide searches can be performed with the NBLAST program, with a score of 100 and a word length of 12, to obtain nucleotide sequences homologous to the nucleic acid molecule of the present disclosure. BLAST protein searches can be performed using the XBLAST program, with a score of 50 and a word length of 3, to obtain amino acid sequences homologous to the protein molecule of the present disclosure. When using the BLAST and gapped BLAST programs, the default parameters of the respective programs (e.g., XBLAST and NBLAST) can be used. See www.ncbi.nlm.nih.gov.

The term “nucleic acid” herein includes any compound and/or substance that comprises a polymer of nucleotides. Each nucleotide consists of a base, in particular a purine or pyrimidine base (i.e., cytosine (C), guanine (G), adenine (A), thymine (T), or uracil (U)), a sugar (i.e., deoxyribose or ribose), and a phosphate group. Generally, a nucleic acid molecule is described as a sequence of bases, whereby the bases represent the primary structure (linear structure) of the nucleic acid molecule. The sequence of bases is generally expressed as 5′ to 3′. Herein, the term “nucleic acid molecule” encompasses deoxyribonucleic acid (DNA), including, e.g., complementary DNA (cDNA) and genomic DNA; ribonucleic acid (RNA), in particular messenger RNA (mRNA); the synthetic forms of DNA or RNA; and polymers comprising a mixture of two or more of these molecules. The nucleic acid molecule may be linear or cyclic. Furthermore, the term “nucleic acid molecule” includes both sense and antisense strands, as well as single- and double-stranded forms. Moreover, the nucleic acid molecules described herein may contain naturally occurring or non-naturally occurring nucleotides. Examples of non-naturally occurring nucleotides include modified nucleotide bases having derived sugar or phosphate backbone linkages or chemically modified residues. The nucleic acid molecule also encompasses DNA and RNA molecules suitable for use as vectors for direct expression of the antibodies of the present disclosure in vitro and/or in vivo, e.g., in a host or patient. Such DNA (e.g., cDNA) or RNA (e.g., mRNA) vectors may be unmodified or modified. For example, mRNA can be chemically modified to enhance the stability of the RNA vector and/or the expression of the encoded molecule such that the mRNA can be injected into a subject to produce antibodies in vivo (see, e.g., Stadler et al., Nature Medicine 2017, published online, Jun. 12, 2017, doi: 10.1038/nm.4356 or EP2101823B1).

The “isolated” nucleic acid herein refers to a nucleic acid molecule that has been separated from components of its natural environment. The isolated nucleic acid includes a nucleic acid molecule contained in such a cell: the cell generally contains the nucleic acid molecule, but the nucleic acid molecule is present extrachromosomally or at a chromosomal location different from its natural chromosomal location.

The term “vector” herein refers to a nucleic acid molecule capable of amplifying another nucleic acid to which it has been linked. The term includes vectors that serve as self-replicating nucleic acid structures as well as vectors integrated into the genome of a host cell into which they have been introduced. Certain vectors are capable of directing the expression of nucleic acids to which they are operably linked. Such vectors are called “expression vectors” herein.

The term “host cell” herein refers to a cell into which an exogenous nucleic acid has been introduced, including the progeny of such a cell. Host cells include “transformants” and “transformed cells”, which include primary transformed cells and progenies derived therefrom, regardless of the number of passages. Progenies may not be exactly the same as parent cells in terms of nucleic acid content, and may contain mutations. Mutant progenies having the same function or biological activity that are screened or selected from the primary transformed cells are included herein.

The term “pharmaceutical composition” herein refers to a formulation that exists in a form allowing the biological activity of the active ingredient contained therein to be effective and does not contain additional ingredients having unacceptable toxicity to a subject to which the pharmaceutical composition is administered.

The term “pharmaceutically acceptable carrier” herein includes any and all solvents, dispersion media, coatings, surfactants, antioxidants, preservatives (e.g., antibacterial agents and antifungal agents), isotonic agents, absorption retardants, salts, preservatives, drug stabilizers, binders, excipients, disintegrants, lubricants, sweeteners, flavorants, dyes, and the like, and combinations thereof, as known to those skilled in the art. Except insofar as any conventional carrier is incompatible with the active ingredient, its use in the therapeutic or pharmaceutical compositions is contemplated.

The term “treat” used herein refers to surgical or therapeutic treatment for the purpose of preventing or slowing (reducing) the progression of an undesired physiological or pathological change, e.g., cancer or tumor. Beneficial or desired clinical results include, but are not limited to, alleviation of symptoms, decrease of severity of disease, stabilization (i.e., not worsening) of state of disease, delay or slowing of disease progression, amelioration or palliation of state of disease, and remission of state of disease (whether partial or total), whether detectable or undetectable. Subjects in need of treatment include those already with a disorder or disease, as well as those who are susceptible to a disorder or disease or those who intend to prevent a disorder or disease. When referring to terms such as slowing, alleviation, decrease, palliation, and remission, their meanings also include elimination, disappearance, nonoccurrence, etc.

The term “subject” herein refers to an organism that receives the treatment for a particular disease or disorder described herein. Illustratively, the “subject” includes mammals, such as humans, primates (e.g., monkey), or non-primate mammals, that receive the treatment for a disease or disorder.

The term “effective amount” herein refers to an amount of a therapeutic agent that is effective in preventing or alleviating symptoms of a disease or the progression of the disease when administered to a cell, tissue, or subject alone or in combination with another therapeutic agent. “Effective amount” also refers to an amount of a compound that is sufficient to alleviate symptoms, e.g., to treat, cure, prevent, or alleviate related medical disorders, or to increase the rates at which such disorders are treated, cured, prevented, or alleviated. When the active ingredient is administered alone to an individual, a therapeutically effective dose refers to the amount of the ingredient alone. When a combination is used, a therapeutically effective dose refers to the combined amounts of the active ingredients that produce the therapeutic effect, whether administered in combination, sequentially, or simultaneously.

The term “cancer” herein refers to or describes a physiological condition in mammals that is typically characterized by unregulated cell growth. Included in this definition are benign and malignant cancers. The term “tumor” or “neoplasm” herein refers to all neoplastic cell growth and proliferation, whether malignant or benign, and all pre-cancerous and cancerous cells and tissues. The terms “cancer” and “tumor” are not mutually exclusive when referred to herein.

The term “EC50” herein refers to the half maximum effective concentration, which includes the antibody concentration that induces a halfway response between the baseline and maximum after a specified exposure time. EC50 essentially represents the antibody concentration at which 50% of the maximal effect is observed, and can be measured by methods known in the art.

### DETAILED DESCRIPTION

The present disclosure provides a nanobody capable of specifically binding to G protein-coupled receptor class C group 5 member D (GPRC5D). Compared with full-length antibodies, the nanobody is capable of better binding to such antigens as GPRC5D that are difficult to bind to. Meanwhile, a novel treatment means is provided for patients with no expression or low expression of BCMA and with poor prognosis for BCMA-targeted treatment.

The present disclosure further provides an antibody or an antigen-binding fragment thereof that specifically binds to GPRC5D, a multispecific antigen-binding molecule, a nucleic acid fragment, a vector, a host cell, a preparation method, a pharmaceutical composition, pharmaceutical use, and a method for treating a tumor or cancer (e.g., B cell lymphoma or multiple myeloma).

In a first aspect, the present disclosure provides an antibody or an antigen-binding fragment thereof that specifically binds to human or monkey GPRC5D, which comprises a CDR1, a CDR2, and a CDR3, wherein the CDR1, the CDR2, and the CDR3 comprise an HCDR1, an HCDR2, and an HCDR3, respectively, of a VHH domain set forth in any one of SEQ ID NOs: 10-13, 50-75, 76-77, and 96-99.

In some specific embodiments, the HCDR1, the HCDR2, and the HCDR3 are determined according to IMGT, Kabat, or Chothia numbering scheme and are selected from any one of the following:
(1) based on the IMGT, Kabat, or Chothia numbering scheme, the HCDR1, the HCDR2, and the HCDR3 comprise the amino acid sequences of SEQ ID NOs: 14, 15, and 16, respectively, the amino acid sequences of SEQ ID NOs: 26, 27, and 28, respectively, or the amino acid sequences of SEQ ID NOs: 38, 39, and 28, respectively;
(2) based on the IMGT, Kabat, or Chothia numbering scheme, the HCDR1, the HCDR2, and the HCDR3 comprise the amino acid sequences of SEQ ID NOs: 17, 18, and 19, respectively, the amino acid sequences of SEQ ID NOs: 29, 30, and 31, respectively, or the amino acid sequences of SEQ ID NOs: 40, 41, and 31, respectively;
(3) based on the IMGT, Kabat, or Chothia numbering scheme, the HCDR1, the HCDR2, and the HCDR3 comprise the amino acid sequences of SEQ ID NOs: 20, 21, and 22, respectively, the amino acid sequences of SEQ ID NOs: 32, 33, and 34, respectively, or the amino acid sequences of SEQ ID NOs: 42, 43, and 34, respectively;
(4) based on the IMGT, Kabat, or Chothia numbering scheme, the HCDR1, the HCDR2, and the HCDR3 comprise the amino acid sequences of SEQ ID NOs: 23, 24, and 25, respectively, the amino acid sequences of SEQ ID NOs: 35, 36, and 37, respectively, or the amino acid sequences of SEQ ID NOs: 44, 45, and 37, respectively;
(5) based on the IMGT, Kabat, or Chothia numbering scheme, the HCDR1, the HCDR2, and the HCDR3 comprise the amino acid sequences of SEQ ID NOs: 78, 79, and 80, respectively, the amino acid sequences of SEQ ID NOs: 84, 85, and 86, respectively, or the amino acid sequences of SEQ ID NOs: 90, 91, and 86, respectively;
(6) based on the IMGT, Kabat, or Chothia numbering scheme, the HCDR1, the HCDR2, and the HCDR3 comprise the amino acid sequences of SEQ ID NOs: 81, 82, and 83, respectively, the amino acid sequences of SEQ ID NOs: 87, 88, and 89, respectively, or the amino acid sequences of SEQ ID NOs: 92, 93, and 89, respectively; or
(7) amino acid sequences having 1, 2, or 3 mutations compared with the HCDR1, the HCDR2, and/or the HCDR3 described in (1)-(6), wherein the mutation is selected from an insertion, a deletion, and/or a substitution, and the substitution is preferably a conservative amino acid substitution.

In some specific embodiments, the CDR1, the CDR2, and/or the CDR3 comprise amino acid sequences having at least 80, 85%, 90%, 91%, 92%, 93%, 94%, 95%, 96%, 97%, 98%, 99%, or 100% identity to the HCDR1, the HCDR2, and/or the HCDR3, respectively, described above.

In some specific embodiments, the antibody or the antigen-binding fragment thereof is a single domain antibody, and the single domain antibody comprises the CDR1, the CDR2, and the CDR3 described above.

In some specific embodiments, the single domain antibody comprises the amino acid sequence set forth in any one of SEQ ID NOs: 10-13, 50-75, 76-77, and 96-99.

Optionally, the single domain antibody comprises an amino acid sequence having at most 20, 19, 18, 17, 16, 15, 14, 13, 12, 11, 10, 9, 8, 7, 6, 5, 4, 3, 2, or 1 mutation compared with the sequence set forth in any one of SEQ ID NOs: 10-13, 50-75, 76-77, and 96-99, wherein the mutation is selected from an insertion, a deletion, and/or a substitution, and the substitution is preferably a conservative amino acid substitution.

Optionally, the single domain antibody comprises an amino acid sequence having at least 80%, 85%, 90%, 91%, 92%, 93%, 94%, 95%, 96%, 97%, 98%, 99%, or 100% identity to the sequence set forth in any one of SEQ ID NOs: 10-13, 50-75, 76-77, and 96-99.

In some specific embodiments, the antibody comprises an FR region in the VHH domain set forth in any one of SEQ ID NOs: 10-13, 50-75, 76-77, and 96-99.

Optionally, the antibody comprises an amino acid sequence having at most 15, 14, 13, 12, 11, 10, 9, 8, 7, 6, 5, 4, 3, 2, or 1 mutation compared with the FR region in the VHH domain set forth in any one of SEQ ID NOs: 10-13, 50-75, 76-77, and 96-99, wherein the mutation is selected from an insertion, a deletion, and/or a substitution, and the substitution is preferably a conservative amino acid substitution.

Optionally, the antibody comprises an amino acid sequence having at least 80%, 85%, 90%, 91%, 92%, 93%, 94%, 95%, 96%, 97%, 98%, 99%, or 100% identity to the FR region in the VHH domain set forth in any one of SEQ ID NOs: 10-13, 50-75, 76-77, and 96-99.

In some specific embodiments, the antibody or the antigen-binding fragment thereof is: (1) a chimeric antibody or a fragment thereof, (2) a humanized antibody or a fragment thereof, or (3) a fully human antibody or a fragment thereof.

In some specific embodiments, the antibody or the antigen-binding fragment thereof comprises or does not comprise an antibody heavy chain constant region; optionally, the antibody heavy chain constant region is selected from human, Vicugna pacos, mouse, rat, rabbit, and sheep; optionally, the antibody heavy chain constant region is selected from IgG, IgM, IgA, IgE, and IgD; optionally, the IgG is selected from IgG1, IgG2, IgG3, and IgG4; optionally, the heavy chain constant region is selected from an Fc region, a CH3 region, and an intact heavy chain constant region; preferably, the heavy chain constant region is a human Fc region; preferably, the antibody or the antigen-binding fragment thereof is a heavy chain antibody.

In some specific embodiments, the antibody or the antigen-binding fragment thereof is further conjugated to a therapeutic agent or a tracer, preferably, the therapeutic agent is selected from a radioisotope, a chemotherapeutic agent, an immunomodulator, and combinations thereof, preferably, the tracer is selected from a radiocontrast medium, a paramagnetic ion, a metal, a fluorescent label, a chemiluminescent label, an ultrasound contrast agent, a photosensitizer, and combinations thereof.

In some specific embodiments, the antibody or the antigen-binding fragment thereof is further linked with an additional functional molecule; preferably, the additional functional molecule is selected from one or more of a signal peptide, a protein tag, a cytokine, an angiogenesis inhibitor, and an immune checkpoint inhibitor.

In a second aspect, the present disclosure further provides a multispecific antigen-binding molecule, which comprises the antibody or the antigen-binding fragment thereof described above and an antigen-binding molecule binding to an antigen other than GPRC5D or binding to a GPRC5D epitope different from that of the antibody or the antigen-binding fragment thereof described above; optionally, the antigen other than GPRC5D is selected from: CD3 (preferably CD3ε), CD16, CD137, CD258, PD-1, PD-L1, 4-1BB, CD40, CD64, EGFR, VEGF, HER2, HER1, HER3, IGF-1R, phosphatidylserine (PS), C-Met, HSA, BCMA, MSLN, blood-brain barrier receptor, GPC3, PSMA, CD33, GD2, ROR1, ROR2, FRα, and Gucy2C.

Preferably, the antigen-binding molecule binding to an antigen other than GPRC5D is an antibody or an antigen-binding fragment thereof.

Preferably, the multispecific antigen-binding molecule may be bispecific, trispecific, or tetraspecific.

preferably, the multispecific antigen-binding molecule may be divalent, trivalent, tetravalent, pentavalent, or hexavalent.

In a third aspect, the present disclosure further provides an isolated nucleic acid fragment encoding the antibody or the antigen-binding fragment thereof described above or the multispecific antigen-binding molecule described above.

In a fourth aspect, the present disclosure further provides a vector, which comprises the nucleic acid fragment described above.

In a fifth aspect, the present disclosure further provides a host cell, which comprises the nucleic acid fragment described above or the vector described above; preferably, the cell is a prokaryotic cell or a eukaryotic cell, such as a bacterium (E. coli), a fungus (yeast), an insect cell, or a mammalian cell, e.g., a CHO cell line or a 293T cell line.

In a sixth aspect, the present disclosure further provides a method for preparing the antibody or the antigen-binding fragment described above or the multispecific antigen-binding molecule described above, which comprises culturing the host cell described above, and isolating an antibody or an antigen-binding fragment thereof or a multispecific antigen-binding molecule expressed by the host cell.

In a seventh aspect, the present disclosure further provides a pharmaceutical composition, which comprises the antibody or the antigen-binding fragment thereof described above, the multispecific antigen-binding molecule described above, the nucleic acid fragment described above, the vector described above, or a product prepared by the method described above; optionally, the pharmaceutical composition further comprises a pharmaceutically acceptable carrier, a diluent, or an adjuvant, and optionally, the pharmaceutical composition further comprises an additional antineoplastic agent.

In an eighth aspect, the present disclosure further provides a method for treating a tumor or cancer, which comprises administering to a subject an effective amount of the antibody or the antigen-binding fragment thereof described above, the multispecific antigen-binding molecule described above, the nucleic acid fragment described above, the vector described above, a product prepared by the method described above, or the pharmaceutical composition described above; the tumor or cancer is preferably B cell lymphoma and more preferably multiple myeloma (MM).

In a ninth aspect, the present disclosure further provides use of the antibody or the antigen-binding fragment thereof described above, the multispecific antigen-binding molecule described above, the nucleic acid fragment described above, the vector described above, a product prepared by the method described above, or the pharmaceutical composition described above in preparing a medicament for treating a tumor or cancer; the tumor or cancer is preferably B cell lymphoma and more preferably multiple myeloma (MM).

In a tenth aspect, the present disclosure further provides the antibody or the antigen-binding fragment thereof described above, the multispecific antigen-binding molecule described above, the nucleic acid fragment described above, the vector described above, a product prepared by the method described above, or the pharmaceutical composition described above for use in treating a tumor or cancer; the tumor or cancer is preferably B cell lymphoma and more preferably multiple myeloma (MM).

In an eleventh aspect, the present disclosure further provides a kit, which comprises the antibody or the antigen-binding fragment thereof described above, the multispecific antigen-binding molecule described above, the nucleic acid fragment described above, the vector described above, a product prepared by the method described above, or the pharmaceutical composition described above.

In a twelfth aspect, the present disclosure further provides a method for detecting expression of GPRC5D in a biological sample, which comprises contacting the biological sample with the antibody or the antigen-binding fragment thereof described above under conditions that allow the antibody or the antigen-binding fragment thereof and GPRC5D to form a complex; preferably, the method further comprises detecting formation of the complex and indicating presence or an expression level of GPRC5D in the sample.

In a thirteenth aspect, the present disclosure further provides use of the antibody or the antigen-binding fragment thereof described above in preparing a GPRC5D detection reagent.

### EXAMPLES

The present disclosure will be further described with reference to specific examples, and the advantages and features of the present disclosure will become more apparent with the description. Experimental procedures without specified conditions in the examples are conducted according to conventional conditions or conditions recommended by the manufacturers. Reagents or instruments without specified manufacturers used herein are conventional products that are commercially available.

The examples are exemplary only and do not limit the scope of the present disclosure in any way. It will be understood by those skilled in the art that various changes or substitutions in form and details may be made to the technical solutions of the present disclosure without departing from the spirit and scope of the present disclosure, and that these changes and substitutions shall fall within the scope of the present disclosure.

### Example 1. Construction of Overexpression Cell Lines, Preparation of Control Antibodies and Purification Method Thereof

### 1.1 Recombinant Expression of GPRC5D Control Antibodies

The control antibodies used in the examples were all derived from published patent sequences, and the sequences of antibodies JNJ7564 and 5F11 were derived from published patents WO2020148677A1 and WO2019154890A1. Unless otherwise indicated, the JNJ7564 and 5F11 control antibodies were recombinantly expressed using the human IgG1+κ subtype. The nanobodies and humanized antibodies thereof used in the examples were all recombinantly expressed using the human Fc fusion form. The specific sequence information of the antibodies is shown in Table 1.

TABLE 1
Sequence listing of control antibodies

| | Sequence | |
|---|---|---|
| Sequence | No. | Amino acid sequence |
| JNJ7564 VH | SEQ ID | QVQLVQSGAEVKKPGASVKVSCKASGYSFTGYTMNWVRQAPG |
| | NO: 1 | QGLEWMGLINPYNSDTNYAQKLQGRVTMTTDTSTSTAYMELRS |
| | | LRSDDTAVYYCARVALRVALDYWGQGTLVTVSS |
| JNJ7564 VL | SEQ ID | DIQMTQSPSSLSASVGDRVTITCKASQNVATHVGWYQQKPGKA |
| | NO: 2 | PKRLIYSASYRYSGVPSRFSGSGSGTEFTLTISNLQPEDFATYYCQ |
| | | QYNRYPYTFGQGTKLEIK |
| SF11 VH | SEQ ID | EVQLVESGGGLVQPGRSLKLSCAASGFSFSNYGMAWVRQAATK |
| | NO: 3 | GLEWVASISTGGGNTYYRDSVKGRFIVSRDNAKNTQYLQMDSL |
| | | RSEDTATYYCTRHDRGGLYWGQGVMVTVSS |
| SF11 VL | SEQ ID | DIVMTQAPLSVSVTPGESASISCRSSKSLLHSNGITYVYWYFQKP |
| | NO: 4 | GKSPQVLIYRMSNLASGVPDRESGSGSETDFTLKISRVEAEDVGI |
| | | YHCGQLLENPYTFGAGTELELK |
| Human IgG1 | SEQ ID | ASTKGPSVFPLAPSSKSTSGGTAALGCLVKDYFPEPVTVSWNSG |
| heavy chain | NO: 5 | ALTSGVHTFPAVLQSSGLYSLSSVVTVPSSSLGTQTYICNVNHKP |
| constant | | SNTKVDKKVEPKSCDKTHTCPPCPAPELLGGPSVFLFPPKPKDT |
| region | | LMISRTPEVTCVVVDVSHEDPEVKFNWYVDGVEVHNAKTKPR |
| | | EEQYNSTYRVVSVLTVLHQDWLNGKEYKCKVSNKALPAPIEKT |
| | | ISKAKGQPREPQVYTLPPSREEMTKNQVSLTCLVKGFYPSDIAVE |
| | | WESNGQPENNYKTTPPVLDSDGSFFLYSKLTVDKSRWQQGNVF |
| | | SCSVMHEALHNHYTQKSLSLSPGK |
| Human IgG1 | SEQ ID | RTVAAPSVFIFPPSDEQLKSGTASVVCLLNNFYPREAKVQWKVD |
| light chain | NO: 6 | NALQSGNSQESVTEQDSKDSTYSLSSTLTLSKADYEKHKVYACE |
| constant | | VTHQGLSSPVTKSFNRGEC |
| region | | |
| Human IgG1 | SEQ ID | EPKSSDKTHTCPPCPAPELLGGPSVFLFPPKPKDTLMISRTPEVT |
| Fc (C220S) | NO: 7 | CVVVDVSHEDPEVKFNWYVDGVEVHNAKTKPREEQYNSTYRV |
| | | VSVLTVLHQDWLNGKEYKCKVSNKALPAPIEKTISKAKGQPREP |
| | | QVYTLPPSREEMTKNQVSLTCLVKGFYPSDIAVEWESNGQPENN |
| | | YKTTPPVLDSDGSFFLYSKLTVDKSRWQQGNVFSCSVMHEALH |
| | | NHYTQKSLSLSPGK |

Note:
In the human IgG1 Fc sequence, the hinge region sequence is bolded and contains the C220S mutation, which is underlined.

### 1.2 Purification of Control Antibodies by Protein a Affinity Chromatography

The obtained antibody sequences were each cloned into a eukaryotic expression vector pTT5 (Youbio, VT2202). Expi293F cells (Gibco, A14527) were transiently transfected with the vector by using PEI (Polysciences, 24765-1), cultured for 7 days, and then centrifuged at high speed to collect the cell culture supernatant expressing the antibodies. The Protein A (Bestchrom, AA0273) protein chromatographic column was washed with 3-5 column volumes of 0.1 M NaOH and then washed with 3-5 column volumes of pure water. The chromatographic column was equilibrated using 3-5 column volumes of 1×PBS (pH 7.4) buffer system as the equilibration buffer. The cell supernatant was loaded on the column at a low flow rate for binding, with the flow rate controlled to allow for a retention time of about 1 min or longer. After the binding was completed, the chromatographic column was washed with 3-5 column volumes of 1×PBS (pH 7.4) until the UV absorbance fell back to baseline. The sample was eluted with a 50 mM citric acid/sodium citrate (pH 3.0-3.5) buffer, the elution peaks were collected based on UV detection, and the eluted product was rapidly adjusted to pH 5-6 with 1 M Tris-HCl (pH 8.0) and temporarily stored. For the eluted product, solution exchange could be performed by methods well known to those skilled in the art, such as ultrafiltration concentration using an ultrafiltration tube and exchange of the solution to a desired buffer system, or exchange with a desired buffer system by molecular exclusion such as G-25 desalination, or removal of polymer components from the eluted product using a high-resolution molecular exclusion column such as Superdex200 to improve sample purity. After protein A protein affinity purification, antibodies eluted from the chromatographic column with a human Fc tag were collected to give corresponding control antibodies. The resulting control antibodies were designated as JNJ7564-hIgG1 and 5F11-hIgG1.

### 1.3 Construction of Overexpression Cell Lines

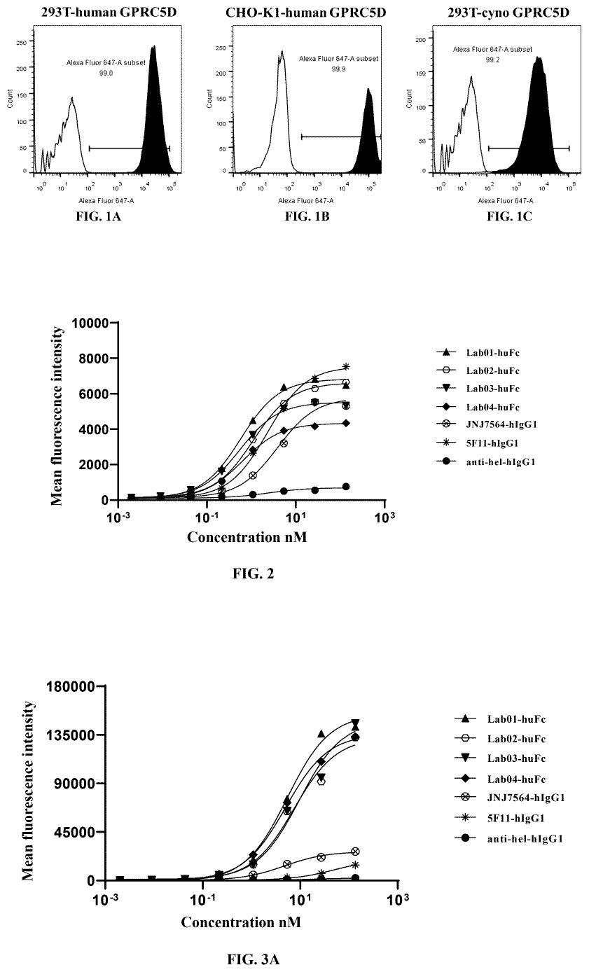

The nucleotide sequence encoding the full-length amino acid sequence of human GPRC5D (UniProt: Q9NZD1, SEQ ID NO: 8) was cloned into a pLVX lentiviral vector (Youbio, VT1465), and viral particles were prepared in HEK293T (purchased from ATCC) cells. After lentiviral infection of the HEK293T cell line, the cells were subjected to selective culturing for 2 weeks in a DMEM medium containing 2 μg/mL puromycin and 10% (w/w) fetal bovine serum and then detected with a human anti-human GPRC5D antibody (JNJ7564-hIgG1, prepared as described in section 1.2) and a goat anti-human IgG (H+L) antibody (Jackson, 109605088) in a flow cytometer FACS CantoII (purchased from BD Biosciences). Cells with high expression level and single peak shape were expanded, and the expanded cells were retested by flow cytometry. Positive cell populations with good growth vigor, high fluorescence intensity, and good uniformity were selected for further expansion and then cryopreserved in liquid nitrogen. The resulting cell strain was named 293T-human GPRC5D, and the expression of the cell line as measured by FACS is shown in FIG. 1A. CHOK1 cells were stably transformed by the same method, and the resulting cell strain was named CHO-K1-human GPRC5D. The expression of the cell line as measured by FACS is shown in FIG. 1B. The nucleotide sequence encoding the full-length amino acid sequence of cyno GPRC5D (NCBI: XP_005570249.1, SEQ ID NO: 9) was cloned into a pLVX lentiviral vector, and the same cell transfection procedure was used to prepare a cell line overexpressing monkey GPRC5D. The resulting cell strain was named 293T-cyno GPRC5D, and the expression of the cell line as measured by FACS is shown in FIG. 1C.

Full-length amino acid sequence of human GPRC5D
(Uniprot: Q9NZD1, SEQ ID NO: 8):
MYKDCIESTGDYFLLCDAEGPWGIILESLAILGIVVTILLLLAFLFLMRK
IQDCSQWNVLPTQLLFLLSVLGLFGLAFAFIIELNQQTAPVRYFLFGVLF
ALCFSCLLAHASNLVKLVRGCVSFSWTTILCIAIGCSLLQIIIATEYVTL
IMTRGMMFVNMTPCQLNVDFVVLLVYVLFLMALTFFVSKATFCGPCENWK
QHGRLIFITVLFSIIIWVVWISMLLRGNPQFQRQPQWDDPVVCIALVTNA
WVFLLLYIVPELCILYRSCRQECPLQGNACPVTAYQHSFQVENQELSRAR
DSDGAEEDVALTSYGTPIQPQTVDPTQECFIPQAKLSPQQDAGGV
Full-length amino acid sequence of Cyno GPRC5D
(NCBI: XP_005570249.1, SEQ ID NO: 9):
MYKDCIESTGDYFLPCDSEGPWGIVLESLAILGIVVTILLLLAFLFLMRK
IQDCSQWNVLPTQLLFLLSVLGLFGLAFAFIIQLNQQTAPVRYFLFGVLF
ALCFSCLLAHASNLVKLVRGRVSFSWTTILCIAIGCSLLQVIIAIEYVTL
IMTRGMMFVHMTPYQLNVDFVVLLVYVLFLMALTFFVSKATFCGPCENWK
QHGRLIFITVLFSIIIWVVWISMLLRGNPQFQRQPQWDDPVVCIALVTNA
WVFLLLYIVPELCILYRSCRQECPSQGHACPVTAYQRSFQVENQELSRAR
DSDGAEEDVALTSFGTPIQPQTVDPTQECFIPRAKLSPQQDAGV

### Example 2. Preparation of Nanobodies Against GPRC5D Proteins

### 2.1 Animal Immunization and Serum Titer Assay

Two camels of about 3 years old were selected, and 5 mL of blood was collected before immunization and used as the negative control serum. In the primary immunization, 0.2 mg of human GPRC5D protein (Kactus Biosystems, GPR-HM05P) was injected subcutaneously at multiple sites into the neck for immunization. After two weeks, a second immunization was performed. 0.1 mg of human GPRC5D protein was injected subcutaneously at multiple sites into the neck for immunization. After one week, the serum was taken for titer assay. The titer and specificity of the antibody in the serum against human and monkey GPRC5D proteins were assayed using the enzyme-linked immunosorbent assay (ELISA). After three protein immunizations, four immunizations were performed using the CHO-K1-human GPRC5D overexpression cell line, and the amount of each cell immunization was 2×10⁷ cells per camel. The results of the serum titers of the sixth immunization are shown in Table 2, and the data in the table are OD_{450 mm} values.

TABLE 2
Serum antibody titer of animals after
immunization assayed by ELISA

Batch

| | Animal 1 | Animal 2 | Animal 1 | Animal 2 |
|---|---|---|---|---|
| | Human | Human | Monkey | Monkey |
| Dilution | GPRC5D | GPRC5D | GPRC5D | GPRC5D |
| ratio | protein | protein | protein | protein |
| 100 | 1.037 | 1.093 | 1.448 | 1.107 |
| 300 | 0.745 | 0.699 | 0.904 | 0.868 |
| 900 | 0.464 | 0.394 | 0.715 | 0.569 |
| 2700 | 0.259 | 0.273 | 0.373 | 0.306 |
| 8100 | 0.158 | 0.163 | 0.187 | 0.179 |
| 24300 | 0.175 | 0.135 | 0.112 | 0.111 |
| 72900 | 0.146 | 0.125 | 0.1 | 0.102 |
| 218700 | 0.13 | 0.12 | 0.087 | 0.089 |
| 656100 | 0.174 | 0.172 | 0.085 | 0.079 |
| Blank | 0.101 | 0.095 | 0.088 | 0.069 |

### 2.2 Construction of Nanobody Phage Library

After the immunization was completed, 50 mL of peripheral blood was collected, and PBMCs were isolated using a lymphocyte isolation medium. The total RNA was extracted using the Trizol reagent, and cDNA was obtained according to the instructions of the reverse transcription kit Prime Script™ II 1st Strand cDNA Synthesis Kit (Takara, 6210A). The nanobody (VHH) fragments were amplified by nested PCR. After being purified, the PCR product was ligated to the phage display vector pComb3Xss (Chengdu NBbiolab, Co. Ltd., P001) using a restriction endonuclease SfiI (purchased from NEB, R0123L), and the ligation product was purified. The ligation product was then transfected into TG1 (Lucigen, 60502) competent cells by electroporation for 8 times, and 1 mL of SOC medium was immediately added to the cuvette after electroporation to thaw and culture the cells for 1 h, thereby obtaining 8.4 mL of a TG1 cell thawing product in total. The TG1 bacterium solution was serially diluted by 10⁴ times and 10⁵ times. The number of transformants in the nanobody library was determined. The remaining bacterium solution was applied on eight 150 mm ampicillin-resistant plates. The library capacities were calculated to be 4.9×10¹⁰ and 1.7×10¹⁰, respectively. 96 samples were sequenced. It was shown that the insertion rate of the library was about 99%, and no repeated sequence existed, thereby indicating that the construction of the phage library was successful.

### 2.3 Panning of Nanobodies Against GPRC5D

The phage library panning was performed using cells overexpressing human GPRC5D as the antigen. The specific panning strategy is shown in Table 3. In rounds 1, 3, and 5, cells overexpressing human GPRC5D (target cells) were used for panning, and in rounds 2 and 4, negative cells 293T were used for negative selection to remove phages showing non-specific binding. The specific procedure is as follows: Target cell-bound phages selected in round 1 were isolated and amplified overnight. In round 2, the phages selected in round 1 were applied to background cells, and unbound phages were isolated for round 3 panning. Target cell-bound phages selected in round 3 were isolated and amplified overnight, and then round 4 negative panning was performed. The final round was performed, in which the amplified phages selected in round 4 negative selection were divided into two panning samples, one for the target cells and the other for background cell panning.

TABLE 3
Process of phage panning using cells

| Panning round | Cells used as antigen |
|---|---|
| 1 | 293T-human GPRC5D |
| 2 | 293T |
| 3 | 293T-human GPRC5D |
| 4 | 293T |
| 5 | 293T |
| | 293T-human GPRC5D |

The phages were eluted using Gly-HCL. Phages selected in rounds 1, 3, and 5 were added to E. coli TG1 (Lucigen, 60502) in the logarithmic growth phase and incubated at 37° C. for 30 min, thus obtaining a TG1 culture solution. The TG1 culture solution was serially diluted, applied on a plate, and cultured at 37° C. overnight. The number of clones in the 293T-human GPRC5D cell group and that in the control group were counted, and 48 clones were selected and sequenced. Meanwhile, the clones on the plate were washed with 2YT medium (Sangon, A507019), collected, seeded into a fresh medium, and cultured at 37° C. to the logarithmic phase. Helper phages M13KO7 (NEB, N0315S) were added with a ratio of helper phage to E. coli TG1 of 20:1. The mixture was mixed well and left to stand at 37° C. for 30 min. Then, the mixture was cultured at 37° C. for 30 min while shaking and centrifuged at 4000 rpm for 10 min. The cells were collected, a fresh 2YT medium that was ampicillin and kanamycin-resistant was added, and the mixture was cultured at 30° C. overnight while shaking. The mixture was centrifuged at 5000 rpm for 20 min, and the supernatant was collected. A 2.5 M NaCl solution containing 20% PEG6000 was added according to ¼ of the volume of the supernatant, and the mixture was left to stand on ice overnight. The mixture was centrifuged at 5000 rpm and 4° C. for 30 min. The phage pellet was collected and dissolved in a PBS buffer, the resulting solution was centrifuged at 10000 rpm for 10 min to remove residual cell debris, and the phage supernatant was collected for the next round of biopanning. After multiple rounds of panning, the positive phages were continuously enriched in the panning process, so that nanobodies with good specificity and high affinity were selected.

### 2.4 Screening of Phage Clones Binding to Cell by Flow Cytometry Assay (FACS)

Single clones were selected from the plates of round 3 and round 5 and cultured in a 96-well plate. A2YT medium containing an antibiotic and 1% glucose was added at 200 μL/well, and the mixture was cultured at 37° C. and 250 rpm overnight while shaking. 10 μL of the overnight culture was added to 100 μL of 2YT medium containing an antibiotic and 0.5% glucose, and the mixture was cultured until the OD₆₀₀ was 0.4-0.6. Helper phages were added at an infection ratio of 20:1, and the mixture was left to stand at 37° C. for 30 min. Then, the mixture was cultured at 37° C. for 30 min while shaking, 400 μL of 2YT medium containing an antibiotic was added, and the mixture was cultured at 30° C. overnight. The next day, the mixture was centrifuged at 5000 rpm and 4° C. for 20 min, and the resulting supernatant was used for FACS assay of single clones.

The multiple myeloma cell line NCI-H929 (ATCC, CRL-9068) and the overexpression cell strains 293T-human GPRC5D and 293T-cyno GPRC5D were each expanded to 90% confluence in a T-175 cell culture flask. For the overexpression cell lines, the medium was completely removed by pipetting, and then the cells were washed once with the PBS buffer, treated with Trypsin-EDTA (Gibco, 25200072), and collected. After counting, the cells were washed 2 times with PBS phosphate-buffered saline, diluted to 2×10⁶ cells/mL, and added to a 96-well FACS reaction plate at 50 μL/well. 1% (v/v) fetal bovine serum was added to the PBS phosphate-buffered saline as an FACS buffer, and the plate was centrifuged at 1000 rpm and 4° C. and washed 2 times. The phage supernatant was added at 50 μL/well, and the mixture was incubated at 4° C. for 1 h. After the plate was centrifuged and washed 3 times with the FACS buffer, an anti-M13 antibody (Sino Biological, 11973-MM05T) diluted at a ratio of 1:1000 was added at 50 μL/well, and the mixture was incubated at 4° C. for 1 h. After the plate was centrifuged and washed 3 times with the FACS buffer, a fluorescently (Alexa647)-labeled secondary antibody (Jackson Immuno, 115-605-003) was added to each well, and the mixture was incubated at 4° C. for 1 h. The plate was centrifuged and washed 3 times with the FACS buffer. The cells were suspended in 100 μL of FACS buffer, and the suspension was loaded for FACS assay and the results were analyzed.

Through multiple rounds of optimization and screening, 4 positive clones capable of recognizing both human and monkey GPRC5D were obtained and named Lab01, Lab02, Lab03, and Lab04, respectively. The CDRs of their sequences were analyzed using IMGT, KABAT, and Chothia numbering schemes. The corresponding sequence information is shown in Tables 4-5 below, wherein Table 4 shows the amino acid sequences of the 4 nanobody molecules, and Table 5 shows the results of IMGT, Kabat, and Chothia analysis of the CDRs of the 4 nanobody molecules.

TABLE 4
Amino acid sequences of anti-GPRCSD nanobodies

| | Sequence | |
|---|---|---|
| Antibody | No. | Amino acid sequence |
| Lab01 | SEQ ID NO: | EVQLQASGGGSVQAGGSRRLSCVASGYAGGIAYMAWFRQTP |
| | 10 | GKGREGVALIATSGTTSYANSVKGRFTIFRDGADNTVYLEMSS |
| | | LKPEDTATYYCAAKSRLGWPGLETSQFKYWGQGTQVTVSS |
| Lab02 | SEQ ID NO: | DVQLQASGGGSVQAGESLRLSCAASGYLYMGWFRQAPGKER |
| | 11 | EGVAAIVTQSGLTGYADSVKGRFTISQDNAENRIYLQMNSLKP |
| | | EDTAMYYCAAKSRLGWTLSPSAYNYWGRGTQVTVSS |
| Lab03 | SEQ ID NO: | DVQLQASGGGSVQAGGSLRLSCAASGDTANCIGWFRQAPGK |
| | 12 | GREGVAAIYTGLGNTYYANSAKGRFTIAQDNAKNTVYLQMN |
| | | SLKPEDSAMYYCAAGGILGRFCSRYFGQGTQVTVSS |
| Lab04 | SEQ ID NO: | EVQLQASGGGSVQAGGSLRLSCVASGFPYSTSRVGWFRQAPG |
| | 13 | KEREGVATIVPGTGYTYYADSVKGRFTISQDSAKNTVYLQMN |
| | | SLKPEDTAVYYCAAGDRRLLLNLLAPADYFYWGQGTQVTVSS |

TABLE 5
Analysis of CDR sequences of GPRCSD nanobodies by IMGT, KABAT, and
Chothia
IMGT Analysis

| | Sequence | | Sequence | | Sequence | |
|---|---|---|---|---|---|---|
| Antibody | No. | CDR1 | No. | CDR2 | No. | CDR3 |
| Lab01 | SEQ ID | GYAGGIA | SEQ ID | IATSGTT | SEQ ID | AAKSRLGWPGLET |
| | NO: 14 | Y | NO: 15 | | NO: 16 | SQFKY |
| Lab02 | SEQ ID | GYLY | SEQ ID | IVTQSGLT | SEQ ID | AAKSRLGWTLSPS |
| | NO: 17 | | NO: 18 | | NO: 19 | AYNY |
| Lab03 | SEQ ID | GDTANC | SEQ ID | IYTGLGNT | SEQ ID | AAGGILGRFCSRY |
| | NO: 20 | | NO: 21 | | NO: 22 | |
| Lab04 | SEQ ID | GFPYSTS | SEQ ID | IVPGTGYT | SEQ ID | AAGDRRLLLNLLA |
| | NO: 23 | R | NO: 24 | | NO: 25 | PADYFY |

Kabat analysis

| | Sequence | | Sequence | | Sequence | |
|---|---|---|---|---|---|---|
| Antibody | No. | CDR1 | No. | CDR2 | No. | CDR3 |
| Lab01 | SEQ ID | IAYMA | SEQ ID | LIATSGTTSY | SEQ ID | KSRLGWPGLETSQ |
| | NO: 26 | | NO: 27 | ANSVKG | NO: 28 | FKY |
| Lab02 | SEQ ID | G | SEQ ID | AIVTQSGLTG | SEQ ID | KSRLGWTLSPSAY |
| | NO: 29 | | NO: 30 | YADSVKG | NO: 31 | NY |
| Lab03 | SEQ ID | CIG | SEQ ID | AIYTGLGNT | SEQ ID | GGILGRFCSRY |
| | NO: 32 | | NO: 33 | YYANSAKG | NO: 34 | |
| Lab04 | SEQ ID | TSRVG | SEQ ID | TIVPGTGYT | SEQ ID | GDRRLLLNLLAPA |
| | NO: 35 | | NO: 36 | YYADSVKG | NO: 37 | DYFY |

Chothia Analysis

| | Sequence | | Sequence | | Sequence | |
|---|---|---|---|---|---|---|
| Antibody | No. | CDR1 | No. | CDR2 | No. | CDR3 |
| Lab01 | SEQ ID | GYAGGIA | SEQ ID | ATSGT | SEQ ID | KSRLGWPGLETSQ |
| | NO: 38 | | NO: 39 | | NO: 28 | FKY |
| Lab02 | SEQ ID | GYL | SEQ ID | VTQSGL | SEQ ID | KSRLGWTLSPSAY |
| | NO: 40 | | NO: 41 | | NO: 31 | NY |
| Lab03 | SEQ ID | GDTAN | SEQ ID | YTGLGN | SEQ ID | GGILGRFCSRY |
| | NO: 42 | | NO: 43 | | NO: 34 | |
| Lab04 | SEQ ID | GFPYSTS | SEQ ID | VPGTGY | SEQ ID | GDRRLLLNLLAPA |
| | NO: 44 | | NO: 45 | | NO: 37 | DYFY |

### Example 3. Expression, Purification and Binding Assay of Recombinant Nanobodies

### 3.1 Expression and Purification of Recombinant Nanobodies

The obtained nanobody sequences were each cloned into a eukaryotic expression vector pTT5 with a hIgG1-Fc tag. Expi293F cells (Gibco, A14527) were transiently transfected with the vector by using PEI, cultured for 7 days, and then centrifuged at high speed to collect the cell culture supernatant expressing the antibodies. The antibodies were purified according to the purification method described in Example 1.2 to obtain corresponding recombinant nanobodies, which were named Lab01-huFc, Lab02-huFc, Lab03-huFc, and Lab04-huFc, respectively. The purified antibodies were each subjected to purity detection using an SEC-HPLC method and then aliquoted for later use after being determined to be acceptable.

### 3.2 Assay on Binding of Recombinant Antibodies to Multiple Myeloma Cell NCI-H929 by Flow Cytometry Assay (FACS)

The multiple myeloma cell NCI-H929 was expanded to the logarithmic growth phase in a T-175 culture flask. After counting, the cells were centrifuged, and the cell pellet was resuspended to 2×10⁶ cells/mL in an FACS buffer (PBS+2% fetal bovine serum) and added to a 96-well FACS reaction plate at 50 μL/well. The plate was centrifuged, and the supernatant was discarded. An antibody sample to be tested (diluted in a 5-fold gradient from a starting concentration of 135 nM) was added at 50 μL/well and uniformly mixed with the cells, and the mixture was incubated at 4° C. for 1 h. After the plate was centrifuged and washed 3 times with a PBS buffer, an Alexa Fluor® 647 AffiniPure Goat Anti-Human IgG and Fcγ fragment specificity-labeled secondary antibody (Jackson, 109-605-098) were added at 50 μL/well, and the mixture was incubated at 4° C. for 1 h. After the plate was centrifuged and washed 3 times again with a PBS buffer and the cells were resuspended in 100 μL of PBS, FACS (BD FACS Canto™) assay and data analysis were performed. The negative control was anti-hel-hIgG1 (Biointron, B117901), an antibody against hen egg lysozyme. Data were analyzed by using the FlowJo software to obtain the mean fluorescence intensity (MFI) of the cells. Then, analysis was performed by the GraphPad Prism8 software, data were fitted, and EC₅₀ values were calculated. As shown in FIG. 2, the recombinant antibodies Lab01-huFc, Lab02-huFc, Lab03-huFc, and Lab04-huFc all can specifically bind to NCI-H929 cells.

### 3.3 Assay on Binding of Recombinant Antibodies to Cells Overexpressing Human and Monkey GPRC5D by Flow Cytometry Assay (FACS)

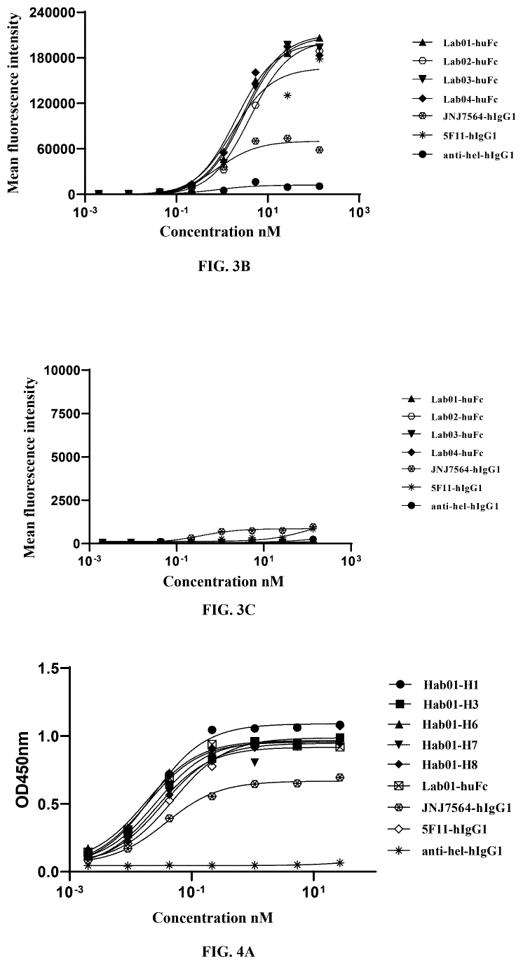

The 293T-human GPRC5D, 293T-cyno GPRC5D, and 293T cells were each expanded to the logarithmic growth phase in a T-175 culture flask, the medium was removed by pipetting, and the cells were washed 2 times with a PBS buffer and digested with trypsin. Then, a complete medium was added to stop the digestion, and the cells were pipetted to form a single cell suspension. After counting, the cells were centrifuged, and the cell pellet was resuspended with an FACS buffer (PBS+2% fetal bovine serum) to 2×10⁶ cells/mL. Then, FACS assay and data analysis were performed according to the method in Example 3.2. As shown in FIGS. 3A-3C, the recombinant antibodies Lab01-huFc, Lab02-huFc, Lab03-huFc, and Lab04-huFc can all efficiently bind to recombinant cells overexpressing human GPRC5D and cyno GPRC5D, and the binding activity is better than that of the positive control antibody. All the recombinant antibodies did not bind to 293T cells and all show good specificity.

### Example 4. Humanization of Nanobodies

### 4.1 Humanization Design for Nanobodies

By alignment with the IMGT database (http://imgt.cines.fr) for human antibody variable region germline gene sequences, germline genes, with high homology to the nanobodies, from heavy chain variable regions were selected as templates, and CDR sequences of the nanobodies based on the IMGT or Kabat naming method were separately grafted into corresponding humanization templates to form variable region sequences in the order of FR1-CDR1-FR2-CDR2-FR3-CDR3-FR4.

The CDR sequences of the Lab01 antibody were defined based on the IMGT scheme, and the humanization templates thereof were IGHV3-7*01 and IGHJ3*01. The Lab01 antibody was renamed HAB01 during the humanization and named HAB01-H after CDR grafting.

The CDR sequences of the Lab02 antibody were defined based on the IMGT scheme, and the humanization templates thereof were IGHV3-30*13 and IGHJ2*01. The Lab02 antibody was renamed HAB02 during the humanization and named HAB02-H after CDR grafting.

The CDR sequences of the Lab03 antibody were defined based on the Kabat scheme, and the humanization templates thereof were IGHV3-7*01 and IGHJ3*01. The Lab03 antibody was renamed HAB03 during the humanization and named HAB03-H after CDR grafting.

The CDR sequences of the Lab04 antibody were defined based on the IMGT scheme, and the humanization templates thereof were IGHV3-7*01 and IGHJ3*01. The Lab04 antibody was renamed HAB04 during the humanization and named HAB04-H after CDR grafting.

CDRs of the antibodies HAB01, HAB02, HAB03, and HAB04 were respectively grafted into the humanization templates thereof to obtain humanized antibodies. The amino acid sequences of the humanization templates and the sequences of the humanized antibodies after CDR grafting are shown in Table 6.

TABLE 6
Specific amino acid sequences of humanization
templates and humanized antibodies

| | Sequence | |
|---|---|---|
| Sequence | No. | Amino acid sequence |
| IGHV3-7*01 | SEQ ID | EVQLVESGGGLVQPGGSLRLSCAASGFT |
| template | NO: 46 | FSSYWMSWVRQAPGKGLEWVANIKQDGS |
| | | EKYYVDSVKGRFTISRDNAKNSLYLQMN |
| | | SLRAEDTAVYYCAR |
| IGHV3- | SEQ ID | QVQLVESGGGVVQPGRSLRLSCAASGFT |
| 30*13 | NO: 47 | FSSYGMHWVRQAPGKGLEWVAVISYDGS |
| template | | NKYYADSVKGRFTISRDNSKNRLYLQMN |
| | | SLRAEDTAVYYCAR |
| IGHJ3*01 | SEQ ID | WGQGTMVTVSS |
| Template | NO: 48 | |
| IGHJ2*01 | SEQ ID | WGRGTLVTVSS |
| Template | NO: 49 | |

### 4.2. Back Mutation Design for HAB01 Humanized Antibodies

Key amino acids in the FR sequence of the HAB01 humanized antibody were back-mutated to amino acids corresponding to the camelid antibody to ensure the original affinity. Details of the mutation points of the specific back mutation (back mutation points were numbered in a natural order) and the specific amino acid sequences are shown in Tables 7-8.

TABLE 7
Mutation points of back mutation of HAB01 humanized antibodies

| Antibody | Mutation point |
|---|---|
| HAB01-H1 | S35A, V37F, L45R, W47G, N73G |
| Graft(IGHV3-7*01) | |
| HAB01-H3 | S35A, V37F, L45R, W47G, N50L, Y58S, N73G |
| Graft(IGHV3-7*01) | |
| HAB01-H6 | E6A, S35A, V37F, L45R, W47G, N50L, Y58S, |
| Graft(IGHV3-7*01) | N73G, L78V |
| HAB01-H7 | S35A, V37F, L45R, W47G, N50L, Y58S, V60A, |
| Graft(IGHV3-7*01) | N73G, L78V |
| HAB01-H8 | S35A, V37F, L45R, W47G, N50L, Y58S, N73G, |
| Graft(IGHV3-7*01) | K75D, L78V |

Note:
Graft(IGHV3-7*01) denotes that the CDRs of the nanobody are grafted into the human germline FR sequences; S35A denotes that S at position 35 of Graft(IGHV3-7*01) is mutated to A, and the same applies to others.

TABLE 8
Amino acid sequences of HAB01 humanized antibodies after back mutation

| Antibody | Sequence No. | Amino acid sequence |
|---|---|---|
| HAB01-H1 | SEQ ID NO: 50 | EVQLVESGGGLVQPGGSLRLSCAASGYAGGIAYMAWFRQAP |
| | | GKGREGVANIATSGTTYYVDSVKGRFTISRDGAKNSLYLQMN |
| | | SLRAEDTAVYYCAAKSRLGWPGLETSQFKYWGQGTMVTVS |
| | | S |
| HAB01-H3 | SEQ ID NO: 51 | EVQLVESGGGLVQPGGSLRLSCAASGYAGGIAYMAWFRQAP |
| | | GKGREGVALIATSGTTSYVDSVKGRFTISRDGAKNSLYLQMN |
| | | SLRAEDTAVYYCAAKSRLGWPGLETSQFKYWGQGTMVTVS |
| | | S |
| HAB01-H6 | SEQ ID NO: 52 | EVQLVASGGGLVQPGGSLRLSCAASGYAGGIAYMAWFRQAP |
| | | GKGREGVALIATSGTTSYVDSVKGRFTISRDGAKNSVYLQMN |
| | | SLRAEDTAVYYCAAKSRLGWPGLETSQFKYWGQGTMVTVS |
| | | S |
| HAB01-H7 | SEQ ID NO: 53 | EVQLVESGGGLVQPGGSLRLSCAASGYAGGIAYMAWFRQAP |
| | | GKGREGVALIATSGTTSYADSVKGRFTISRDGAKNSVYLQMN |
| | | SLRAEDTAVYYCAAKSRLGWPGLETSQFKYWGQGTMVTVS |
| | | S |
| HAB01-H8 | SEQ ID NO: 54 | EVQLVESGGGLVQPGGSLRLSCAASGYAGGIAYMAWFRQAP |
| | | GKGREGVALIATSGTTSYVDSVKGRFTISRDGADNSVYLQMN |
| | | SLRAEDTAVYYCAAKSRLGWPGLETSQFKYWGQGTMVTVS |
| | | S |

The results of the IMGT numbering scheme-based CDR analysis of HAB01 humanized antibodies are shown in Table 9:

TABLE 9
Results of IMGT numbering scheme-based CDR analysis of HAB01 humanized
antibodies

| | Sequence | | Sequence | | Sequence | CDR3 |
|---|---|---|---|---|---|---|
| Antibody | No. | CDR1 | No. | CDR2 | No. | |
| HAB01-H1 | SEQ ID | GYAGGIA | SEQ ID | IATSGTT | SEQ ID | AAKSRLGWPG |
| HAB01-H3 | NO: 14 | Y | NO: 15 | | NO: 16 | LETSQFKY |
| HAB01-H6 | | | | | | |
| HAB01-H7 | | | | | | |
| HAB01-H8 | | | | | | |

### 4.3. Back Mutation Design for HAB02 Humanized Antibodies

Key amino acids in the FR sequence of the HAB02 humanized antibody were back-mutated to amino acids corresponding to the camelid antibody to ensure the original affinity. Details of the mutation points of the specific back mutation (back mutation points were numbered in a natural order) and the specific amino acid sequences are shown in Tables 10-11.

TABLE 10
Mutation points of back mutation of HAB02 humanized antibodies

| Antibody | Mutation point |
|---|---|
| HAB02-H1 | Q1E, H31G, V33F, G40E, L41R, W43G |
| Graft(IGHV3-30*13) | |
| HAB02-H2 | Q1E, H31G, V33F, G40E, L41R, W43G, R68Q |
| Graft(IGHV3-30*13) | |
| HAB02-H3 | Q1E, H31G, V33F, G40E, L41R, W43G, V46A, |
| Graft(IGHV3-30*13) | R68Q |
| HAB02-H4 | Q1E, H31G, V33F, G40E, L41R, W43G, V46A, |
| Graft(IGHV3-30*13) | Y55G, R68Q |
| HAB02-H5 | Q1E, H31G, V33F, G40E, L41R, W43G, V46A, |
| Graft(IGHV3-30*13) | Y55G, R68Q, S71A |
| HAB02-H6 | Q1D, H31G, V33F, G40E, L41R, W43G, V46A, |
| Graft(IGHV3-30*13) | Y55G, R68Q |
| HAB02-H7 | Q1E, V5Q, E6A, H31G, V33F, G40E, L41R, |
| Graft(IGHV3-30*13) | W43G, V46A, Y55G, R68Q |
| HAB02-H8 | Q1E, H31G, V33F, G40E, L41R, W43G, V46A, |
| Graft(IGHV3-30*13) | Y55G, R68Q, L75I |

Note:
Graft(IGHV3-30*13) denotes that the CDRs of the nanobody are grafted into the human germline FR sequences; Q1E denotes that Q at position 1 of Graft(IGHV3-30*13) is mutated to E, and the same applies to others.

TABLE 11
Amino acid sequences of HAB02 humanized antibodies after back mutation

| Antibody | Sequence No. | Amino acid sequence |
|---|---|---|
| HAB02-H1 | SEQ ID NO: | EVQLVESGGGVVQPGRSLRLSCAASGYLYMGWFRQAPGK |
| | 55 | EREGVAVIVTQSGLTYYADSVKGRFTISRDNSKNRLYLQMN |
| | | SLRAEDTAVYYCAAKSRLGWTLSPSAYNYWGRGTLVTVSS |
| HAB02-H2 | SEQ ID NO: | EVQLVESGGGVVQPGRSLRLSCAASGYLYMGWFRQAPGK |
| | 56 | EREGVAVIVTQSGLTYYADSVKGRFTISQDNSKNRLYLQMN |
| | | SLRAEDTAVYYCAAKSRLGWTLSPSAYNYWGRGTLVTVSS |
| HAB02-H3 | SEQ ID NO: | EVQLVESGGGVVQPGRSLRLSCAASGYLYMGWFRQAPGK |
| | | EREGVAAIVTQSGLTYYADSVKGRFTISQDNSKNRLYLQMN |
| | 57 | SLRAEDTAVYYCAAKSRLGWTLSPSAYNYWGRGTLVTVSS |
| HAB02-H4 | SEQ ID NO: | EVQLVESGGGVVQPGRSLRLSCAASGYLYMGWFRQAPGK |
| | 58 | EREGVAAIVTQSGLTGYADSVKGRFTISQDNSKNRLYLQMN |
| | | SLRAEDTAVYYCAAKSRLGWTLSPSAYNYWGRGTLVTVSS |
| HAB02-H5 | SEQ ID NO: | EREGVAAIVTQSGLTGYADSVKGRFTISQDNAKNRLYLQM |
| | 59 | EVQLVESGGGVVQPGRSLRLSCAASGYLYMGWFRQAPGK |
| | | NSLRAEDTAVYYCAAKSRLGWTLSPSAYNYWGRGTLVTVS |
| | | S |
| HAB02-H6 | SEQ ID NO: | DVQLVESGGGVVQPGRSLRLSCAASGYLYMGWFRQAPGK |
| | 60 | EREGVAAIVTQSGLTGYADSVKGRFTISQDNSKNRLYLQMN |
| | | SLRAEDTAVYYCAAKSRLGWTLSPSAYNYWGRGTLVTVSS |
| HAB02-H7 | SEQ ID NO: | EVQLQASGGGVVQPGRSLRLSCAASGYLYMGWFRQAPGK |
| | 61 | EREGVAAIVTQSGLTGYADSVKGRFTISQDNSKNRLYLQMN |
| | | SLRAEDTAVYYCAAKSRLGWTLSPSAYNYWGRGTLVTVSS |
| HAB02-H8 | SEQ ID NO: | EVQLVESGGGVVQPGRSLRLSCAASGYLYMGWFRQAPGK |
| | 62 | EREGVAAIVTQSGLTGYADSVKGRFTISQDNSKNRIYLQMN |
| | | SLRAEDTAVYYCAAKSRLGWTLSPSAYNYWGRGTLVTVSS |

The results of the IMGT numbering scheme-based CDR analysis of HAB02 humanized antibodies are shown in Table 12:

TABLE 12
Results of IMGT numbering scheme-based CDR analysis of HAB02 humanized
antibodies

| | Sequence | | Sequence | | Sequence | |
|---|---|---|---|---|---|---|
| Antibody | No. | CDR1 | No. | CDR2 | No. | CDR3 |
| HAB02-H1 | SEQ ID | GYLY | SEQ ID | IVTQSGLT | SEQ ID | AAKSRLGWTL |
| HAB02-H2 | NO: 17 | | NO: 18 | | NO: 19 | SPSAYNY |
| HAB02-H3 | | | | | | |
| HAB02-H4 | | | | | | |
| HAB02-H5 | | | | | | |
| HAB02-H6 | | | | | | |
| HAB02-H7 | | | | | | |
| HAB02-H8 | | | | | | |

### 4.4. Back Mutation Design for HAB03 Humanized Antibodies

Key amino acids in the FR sequence of the HAB03 humanized antibody were back-mutated to amino acids corresponding to the camelid antibody to ensure the original affinity. Details of the mutation points of the specific back mutation (back mutation points were numbered in a natural order) and the specific amino acid sequences are shown in Tables 13-14.

TABLE 13
Mutation points of back mutation of HAB03 humanized antibodies

| Antibody | Mutation point |
|---|---|
| HAB03-H5 | F27D, F29A, S30N, V35F, L43R, W45G, R70Q, |
| Graft(IGHV3-7*01) | L77V, R96A, W108F |
| HAB03-H8 | F27D, F29A, S30N, V35F, W45G, R70Q, L77V, |
| Graft(IGHV3-7*01) | R96A, W108F |
| HAB03-H9 | F27D, F29A, V35F, L43R, W45G, R70Q, L77V, |
| Graft(IGHV3-7*01) | R96A, W108F |
| HAB03-H10 | F27D, F29A, S30N, V35F, L43R, W45G, L77V, |
| Graft(IGHV3-7*01) | R96A, W108F |
| HAB03-H11 | E1D, F27D, F29A, S30N, V35F, L43R, W45G, |
| Graft(IGHV3-7*01) | R70Q, L77V, R96A, W108F |
| HAB03-H12 | E6A, F27D, F29A, S30N, V35F, L43R, W45G, |
| Graft(IGHV3-7*01) | R70Q, L77V, R96A, W108F |

Note:
Graft(IGHV3-7*01) denotes that the CDRs of the nanobody are grafted into the human germline FR sequences; F27D denotes that F at position 27 of Graft(IGHV3-7*01) is mutated to D, and the same applies to others.

TABLE 14
Amino acid sequences of HAB03 humanized antibodies after back mutation

| | Sequence | |
|---|---|---|
| Antibody | No. | Amino acid sequence |
| HAB03-H5 | SEQ ID NO: | EVQLVESGGGLVQPGGSLRLSCAASGDTANCIGWFRQAPGK |
| | 63 | GREGVAAIYTGLGNTYYANSAKGRFTISQDNAKNSVYLQM |
| | | NSLRAEDTAVYYCAAGGILGRFCSRYFGQGTMVTVSS |
| HAB03-H8 | SEQ ID NO: | EVQLVESGGGLVQPGGSLRLSCAASGDTANCIGWFRQAPGK |
| | 64 | GLEGVAAIYTGLGNTYYANSAKGRFTISQDNAKNSVYLQMN |
| | | SLRAEDTAVYYCAAGGILGRFCSRYFGQGTMVTVSS |
| HAB03-H9 | SEQ ID NO: | EVQLVESGGGLVQPGGSLRLSCAASGDTASCIGWFRQAPGK |
| | 65 | GREGVAAIYTGLGNTYYANSAKGRFTISQDNAKNSVYLQM |
| | | NSLRAEDTAVYYCAAGGILGRFCSRYFGQGTMVTVSS |
| HAB03- | SEQ ID NO: | EVQLVESGGGLVQPGGSLRLSCAASGDTANCIGWFRQAPGK |
| H10 | 66 | GREGVAAIYTGLGNTYYANSAKGRFTISRDNAKNSVYLQMN |
| | | SLRAEDTAVYYCAAGGILGRFCSRYFGQGTMVTVSS |
| HAB03- | SEQ ID NO: | DVQLVESGGGLVQPGGSLRLSCAASGDTANCIGWFRQAPGK |
| H11 | 67 | GREGVAAIYTGLGNTYYANSAKGRFTISQDNAKNSVYLQM |
| | | NSLRAEDTAVYYCAAGGILGRFCSRYFGQGTMVTVSS |
| HAB03- | SEQ ID NO: | EVQLVASGGGLVQPGGSLRLSCAASGDTANCIGWFRQAPGK |
| H12 | 68 | GREGVAAIYTGLGNTYYANSAKGRFTISQDNAKNSVYLQM |
| | | NSLRAEDTAVYYCAAGGILGRFCSRYFGQGTMVTVSS |

The results of the Kabat numbering scheme-based CDR analysis of HAB03 humanized antibodies are shown in Table 15:

TABLE 15
Results of Kabat numbering scheme-based CDR analysis of HAB03 humanized
antibodies

| | Sequence | | Sequence | | Sequence | |
|---|---|---|---|---|---|---|
| Antibody | No. | CDR1 | No. | CDR2 | No. | CDR3 |
| HAB03-H5 | SEQ ID | CIG | SEQ ID | AIYTGLGNTY | SEQ ID | GGILGRFCSRY |
| HAB03-H8 | NO: 32 | | NO: 33 | YANSAKG | NO: 34 | |
| HAB03-H9 | | | | | | |
| HAB03-H10 | | | | | | |
| HAB03-H11 | | | | | | |
| HAB03-H12 | | | | | | |

### 4.5. Back Mutation Design for HAB04 Humanized Antibodies

Key amino acids in the FR sequence of the HAB04 humanized antibody were back-mutated to amino acids corresponding to the camelid antibody to ensure the original affinity. Details of the mutation points of the specific back mutation (back mutation points were numbered in a natural order) and the specific amino acid sequences are shown in Tables 16-17.

TABLE 16
Mutation points of back mutation of HAB04 humanized antibodies

| Antibody | Mutation point |
|---|---|
| HAB04-H3 | S35G, V37F, G44E, L45R, W47G, N50T, R72Q |
| Graft(IGHV3-7*01) | |
| HAB04-H4 | S35G, V37F, G44E, L45R, W47G, N50T, R72Q, |
| Graft(IGHV3-7*01) | N74S |
| HAB04-H5 | M34V, S35G, V37F, G44E, L45R, W47G, N50T, |
| Graft(IGHV3-7*01) | R72Q, L79V |
| HAB04-H7 | E6A, S35G, V37F, G44E, L45R, W47G, N50T, |
| Graft(IGHV3-7*01) | R72Q |
| HAB04-H8 | V5Q, A23V, S35G, V37F, G44E, L45R, W47G, |
| Graft(IGHV3-7*01) | N50T, R72Q |
| HAB04-H10 | E6A, S35G, V37F, G44E, L45R, W47G, N50T, |
| Graft(IGHV3-7*01) | R72Q, N74S |
| HAB04-H14 | E6A, S35G, V37F, G44E, L45R, W47G, N50T, |
| Graft(IGHV3-7*01) | V61A, R72Q |

Note:
Graft(IGHV3-7*01) denotes that the CDRs of the nanobody are grafted into the human germline FR sequences; S35G denotes that S at position 35 of Graft(IGHV3-7*01) is mutated to G, and the same applies to others.

TABLE 17
Amino acid sequences of HAB04 humanized antibodies after back mutation

| | Sequence | |
|---|---|---|
| Antibody | No. | Amino acid sequence |
| HAB04- | SEQ ID NO: | EVQLVESGGGLVQPGGSLRLSCAASGFPYSTSRMGWFRQAPG |
| H3 | 69 | KEREGVATIVPGTGYTYYVDSVKGRFTISQDNAKNSLYLQMN |
| | | SLRAEDTAVYYCAAGDRRLLLNLLAPADYFYWGQGTMVTVS |
| | | S |
| HAB04- | SEQ ID NO: | EVQLVESGGGLVQPGGSLRLSCAASGFPYSTSRMGWFRQAPG |
| H4 | 70 | KEREGVATIVPGTGYTYYVDSVKGRFTISQDSAKNSLYLQMNS |
| | | LRAEDTAVYYCAAGDRRLLLNLLAPADYFYWGQGTMVTVSS |
| HAB04- | SEQ ID NO: | EVQLVESGGGLVQPGGSLRLSCAASGFPYSTSRVGWFRQAPG |
| H5 | 71 | KEREGVATIVPGTGYTYYVDSVKGRFTISQDNAKNSVYLQMN |
| | | SLRAEDTAVYYCAAGDRRLLLNLLAPADYFYWGQGTMVTVS |
| | | S |
| HAB04- | SEQ ID NO: | EVQLVASGGGLVQPGGSLRLSCAASGFPYSTSRMGWFRQAPG |
| H7 | 72 | KEREGVATIVPGTGYTYYVDSVKGRFTISQDNAKNSLYLQMN |
| | | SLRAEDTAVYYCAAGDRRLLLNLLAPADYFYWGQGTMVTVS |
| | | S |
| HAB04- | SEQ ID NO: | EVQLQESGGGLVQPGGSLRLSCVASGFPYSTSRMGWFRQAPG |
| H8 | 73 | KEREGVATIVPGTGYTYYVDSVKGRFTISQDNAKNSLYLQMN |
| | | SLRAEDTAVYYCAAGDRRLLLNLLAPADYFYWGQGTMVTVS |
| | | S |
| HAB04- | SEQ ID NO: | EVQLVASGGGLVQPGGSLRLSCAASGFPYSTSRMGWFRQAPG |
| H10 | 74 | KEREGVATIVPGTGYTYYVDSVKGRFTISQDSAKNSLYLQMNS |
| | | LRAEDTAVYYCAAGDRRLLLNLLAPADYFYWGQGTMVTVSS |
| HAB04- | SEQ ID NO: | EVQLVASGGGLVQPGGSLRLSCAASGFPYSTSRMGWFRQAPG |
| H14 | 75 | KEREGVATIVPGTGYTYYADSVKGRFTISQDNAKNSLYLQMN |
| | | SLRAEDTAVYYCAAGDRRLLLNLLAPADYFYWGQGTMVTVS |
| | | S |

The results of the IMGT numbering scheme-based CDR analysis of HAB04 humanized antibodies are shown in Table 18:

TABLE 18
Results of IMGT numbering scheme-based CDR analysis of HAB04 humanized
antibodies

| | Sequence | | Sequence | | Sequence | |
|---|---|---|---|---|---|---|
| Antibody | No. | CDR1 | No. | CDR2 | No. | CDR3 |
| HAB04-H3 | SEQ ID | GFPYSTS | SEQ ID | IVPGTGYT | SEQ ID | AAGDRRLLLN |
| HAB04-H4 | NO: 23 | R | NO: 24 | | NO: 25 | LLAPADYFY |
| HAB04-H5 | | | | | | |
| HAB04-H7 | | | | | | |
| HAB04-H8 | | | | | | |
| HAB04-H10 | | | | | | |
| HAB04-H14 | | | | | | |

### Example 5. Expression, Purification, and Identification of Humanized Nanobodies

### 5.1 Expression and Purification of Humanized Nanobodies

The humanized antibody variable region sequence gene was synthesized and cloned into a pTT5 vector with a human binge region and a hIgG1-Fc constant region sequence to form a VHH-huFc (C220S) expression order, and a plasmid was prepared. The antibody plasmid was transiently transfected into Expi293F cells by using PEI, and after 7 days of culture, the supernatant was collected, and the antibody was prepared by Protein A purification as in Example 1.2.

### 5.2 Assay on Binding of Humanized Antibodies to Human GPRC5D Protein by Enzyme-Linked Immunosorbent Assay (ELISA)

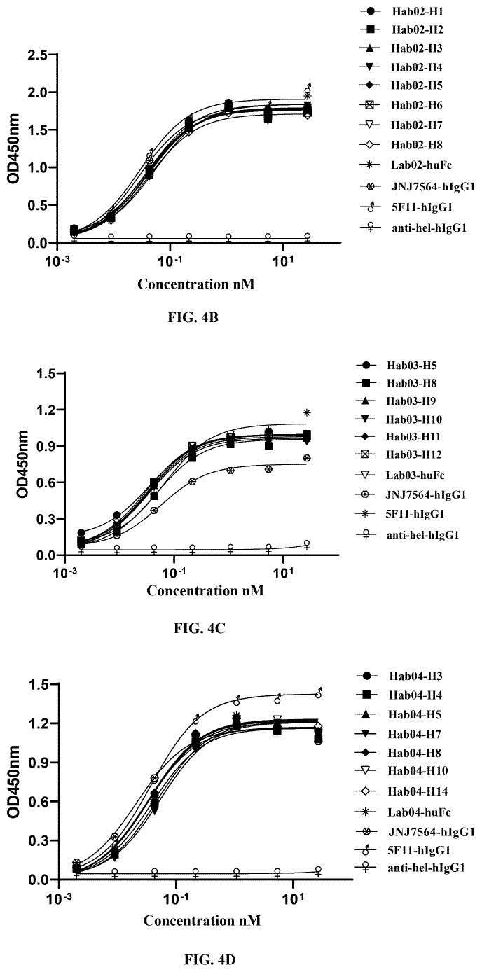

Human GPRC5D protein (Kactus Biosystems, GPR-HM05P) was diluted to a final concentration of 1 μg/mL in PBS, added to a 96-well ELISA plate at 50 μL/well, and then incubated overnight at 4° C. The next day, the plate was washed 2 times with PBST, and a blocking buffer [PBS+2% (w/w) BSA] was added for blocking at room temperature for 2 h. The blocking buffer was then discarded, and the humanized recombinant antibodies (diluted in a 5-fold gradient from a starting concentration of 135 nM) and the positive and negative control antibodies were each added at 50 μL/well. After incubation at 37° C. for 1 h, the plate was washed 3 times with PBST. A horseradish peroxidase (HRP)-labeled Goat Anti-Human secondary antibody (Merck, AP113P) was added. The mixture was incubated at 37° C. for 1 h, and then the plate was washed 5 times with PBST. A TMB substrate was added at 50 μL/well. The mixture was incubated at room temperature for 10 min, and then a stop solution (1.0 M HCl) was added at 50 μL/well. The OD_{450 nm} values were read using an ELISA plate reader (Multimode Plate Reader, EnSight, Perkin Elmer), and the binding activity of the humanized antibodies for human GPRC5D protein is shown in FIGS. 4A-4D. The results show that the humanized antibodies HAB01, HAB02, HAB03, and HAB04 can well bind to human GPRC5D protein, and the specific values of binding activity are shown in Tables 19-22.

TABLE 19
Binding activity of humanized antibody
HAB01 for human GPRC5D protein

| Antibody | Maximum value (OD₄₅₀) | EC50 (nM) |
|---|---|---|
| HAB01_H1 | 1.082 | 0.02303 |
| HAB01_H3 | 0.988 | 0.02589 |
| HAB01_H6 | 0.966 | 0.01777 |
| HAB01_H7 | 0.947 | 0.02639 |
| HAB01_H8 | 0.973 | 0.03365 |
| Lab01-huFc | 0.958 | 0.01821 |
| JNJ7564-hIgG1 | 0.694 | 0.03795 |
| 5F11-hIgG1 | 1.074 | 0.04976 |
| anti-hel-hIgG1 | 0.065 | N/A |

TABLE 20
Binding activity of humanized antibody
HAB02 for human GPRC5D protein

| Antibody | Maximum value (OD₄₅₀) | EC50 (nM) |
|---|---|---|
| HAB02_H1 | 1.854 | 0.04046 |
| HAB02_H2 | 1.820 | 0.03808 |
| HAB02_H3 | 1.813 | 0.04196 |
| HAB02_H4 | 1.843 | 0.03953 |
| HAB02_H5 | 1.862 | 0.04063 |
| HAB02_H6 | 1.795 | 0.03615 |
| HAB02_H7 | 1.794 | 0.03961 |
| HAB02_H8 | 1.721 | 0.04086 |
| Lab02-huFc | 1.951 | 0.05298 |
| JNJ7564-hIgG1 | 1.750 | 0.02796 |
| 5F11-hIgG1 | 2.056 | 0.02948 |
| anti-hel-hIgG1 | 0.058 | N/A |

TABLE 21
Binding activity of humanized antibody
HAB03 for human GPRC5D protein

| Antibody | Maximum value (OD₄₅₀) | EC50 (nM) |
|---|---|---|
| HAB03_H5 | 1.019 | 0.03531 |
| HAB03_H8 | 1.000 | 0.04619 |
| HAB03_H9 | 0.973 | 0.03257 |
| HAB03_H10 | 0.999 | 0.03236 |
| HAB03_H11 | 0.953 | 0.03381 |
| HAB03_H12 | 0.992 | 0.03065 |
| Lab03-huFc | 1.001 | 0.03194 |
| JNJ7564-hIgG1 | 0.800 | 0.05593 |
| 5F11-hIgG1 | 1.175 | 0.06587 |
| anti-hel-hIgG1 | 0.082 | N/A |

TABLE 22
Binding of humanized antibody
HAB04 to human GPRC5D protein

| Antibody | Maximum value (OD₄₅₀) | EC50 (nM) |
|---|---|---|
| HAB04_H3 | 1.243 | 0.03404 |
| HAB04_H4 | 1.182 | 0.03821 |
| HAB04_H5 | 1.239 | 0.03334 |
| HAB04_H7 | 1.231 | 0.05328 |
| HAB04_H8 | 1.181 | 0.03113 |
| HAB04_H10 | 1.228 | 0.04809 |
| HAB04_H14 | 1.235 | 0.03488 |
| Lab04-huFc | 1.262 | 0.03527 |
| JNJ7564-hIgG1 | 1.181 | 0.02115 |
| 5F11-hIgG1 | 1.436 | 0.03707 |
| anti-hel-hIgG1 | 0.061 | N/A |

### 5.3 Assay on Binding of Humanized Antibodies to Multiple Myeloma Cells by Flow Cytometry Assay (FACS)

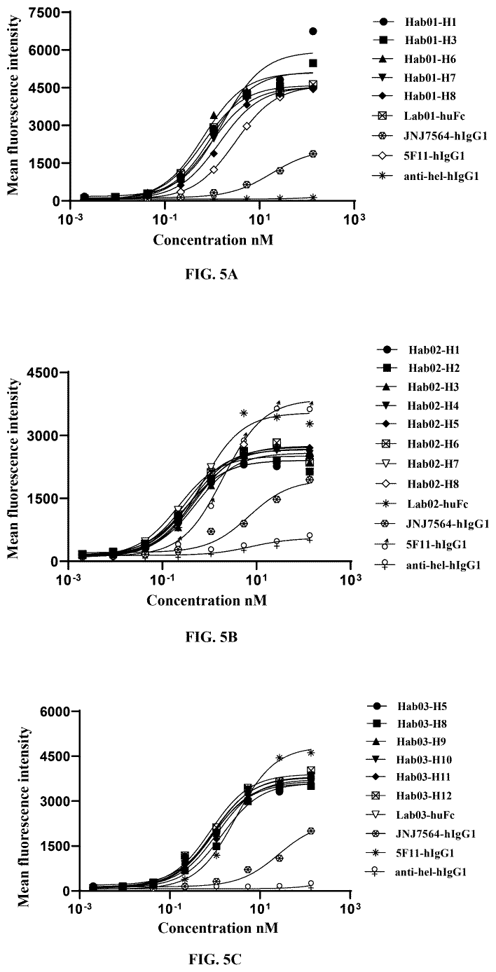

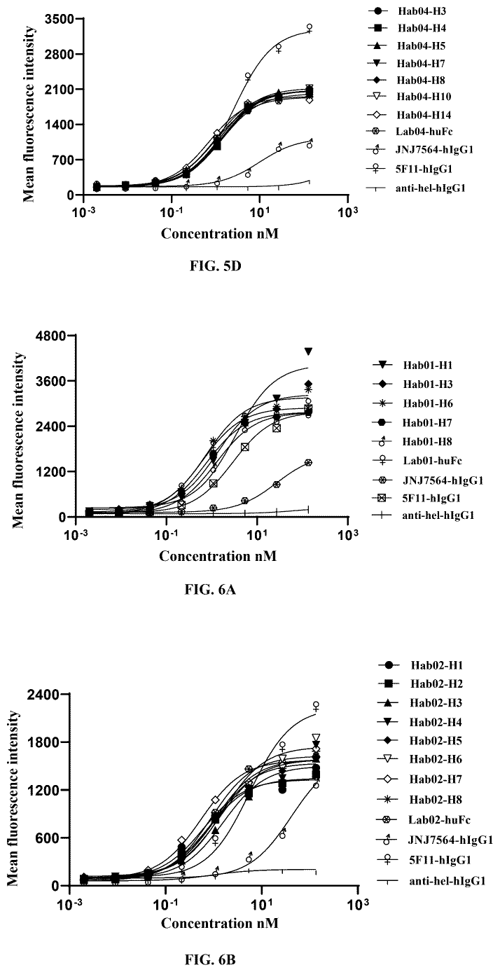

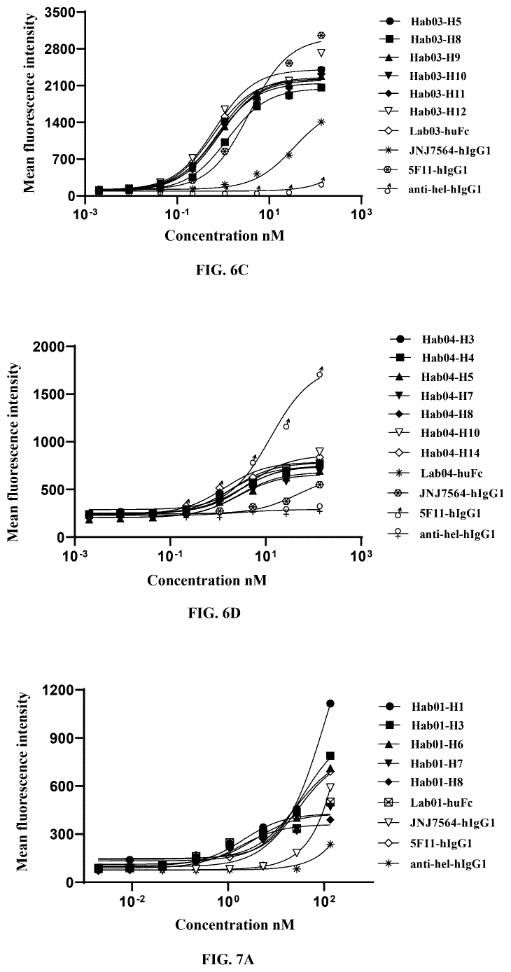

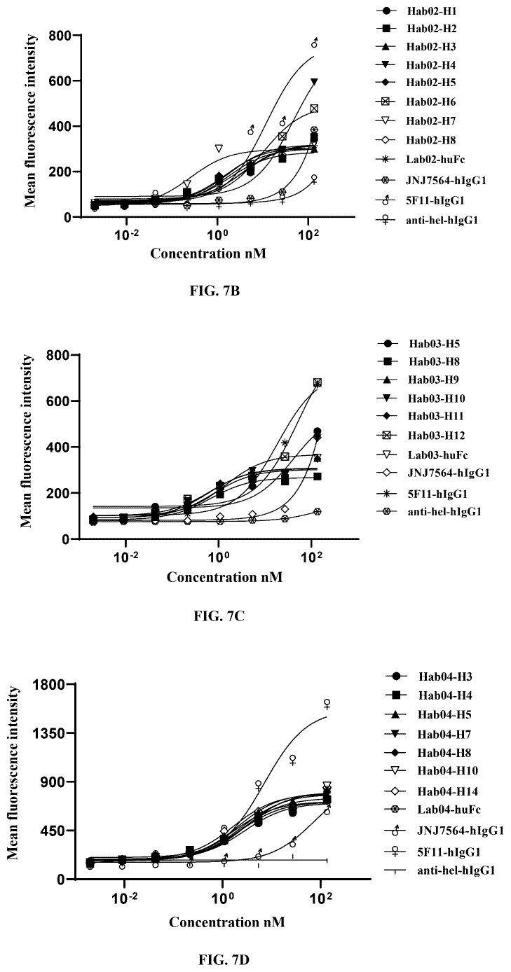

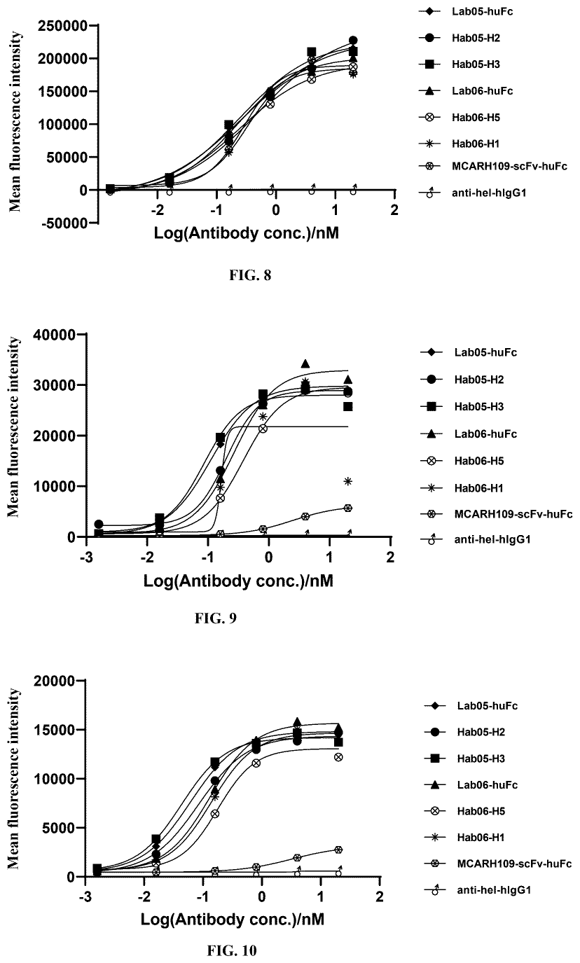

The preparation of the assay cells and the antibodies to be tested and the assay were performed with reference to Example 3.2. The assay results are shown in FIGS. 5-7. The humanized antibodies all have good binding activity for endogenous cells NCI-H929, molp-8 (Cobioer, Nanjing, CBP60562), and RPMI-8226 (ATCC, CCL-155), and specific binding activity and values are shown in Tables 23-34.

TABLE 23
Binding activity of humanized antibody
HAB01 for NCI-H929 cells

| Antibody | Maximum value (MFI) | EC50 (nM) |
|---|---|---|
| HAB01_H1 | 6744 | 1.626 |
| HAB01_H3 | 5477 | 1.019 |
| HAB01_H6 | 5476 | 0.666 |
| HAB01_H7 | 4434 | 0.9159 |
| HAB01_H8 | 4439 | 1.408 |
| Lab01-huFc | 4629 | 0.6529 |
| JNJ7564-hIgG1 | 1871 | 17.88 |
| 5F11-hIgG1 | 4494 | 3.047 |
| anti-hel-hIgG1 | 128 | N/A |

TABLE 24
Binding activity of humanized antibody
HAB02 for NCI-H929 cells

| Antibody | Maximum value (MFI) | EC50 (nM) |
|---|---|---|
| HAB02_H1 | 2516 | 0.312 |
| HAB02_H2 | 2531 | 0.2989 |
| HAB02_H3 | 2704 | 0.51 |
| HAB02_H4 | 2647 | 0.4377 |
| HAB02_H5 | 2707 | 0.5009 |
| HAB02_H6 | 2829 | 0.3169 |
| HAB02_H7 | 2685 | 0.2197 |
| HAB02_H8 | 2787 | 0.4005 |
| Lab02-huFc | 3533 | 0.6719 |
| JNJ7564-hIgG1 | 1946 | 6.97 |
| 5F11-hIgG1 | 3689 | 1.956 |
| anti-hel-hIgG1 | 561 | N/A |

TABLE 25
Binding activity of humanized antibody
HAB03 for NCI-H929 cells

| Antibody | Maximum value (MFI) | EC50 (nM) |
|---|---|---|
| HAB03_H5 | 3815 | 0.8925 |
| HAB03_H8 | 3495 | 1.413 |
| HAB03_H9 | 3714 | 0.8955 |
| HAB03_H10 | 3863 | 0.9443 |
| HAB03_H11 | 3682 | 1.125 |
| HAB03_H12 | 4026 | 0.8084 |
| Lab03-huFc | 3630 | 0.909 |
| JNJ7564-hIgG1 | 2001 | 25.64 |
| 5F11-hIgG1 | 4616 | 2.899 |
| anti-hel-hIgG1 | 181 | N/A |

TABLE 26
Binding activity of humanized antibody
HAB04 for NCI-H929 cells

| | Maximum | EC50 |
|---|---|---|
| Antibody | value (MFI) | (nM) |
| HAB04_H3 | 2099 | 1.349 |
| HAB04_H4 | 2058 | 1.409 |
| HAB04_H5 | 2039 | 1.018 |
| HAB04_H7 | 1972 | 1.345 |
| HAB04_H8 | 2115 | 1.281 |
| HAB04_H10 | 2120 | 1.21 |
| HAB04_H14 | 1925 | 0.711 |
| Lab04-huFc | 1953 | 0.9148 |
| JNJ7564-hIgG1 | 1025 | 9.88 |
| 5F11-hIgG1 | 3302 | 2.685 |
| anti-hel-hIgG1 | 291 | N/A |

TABLE 27
Binding activity of humanized antibody HAB01 for molp-8 cells

| | Maximum | EC50 |
|---|---|---|
| Antibody | value (MFI) | (nM) |
| HAB01_H1 | 4375 | 3.296 |
| HAB01_H3 | 3517 | 1.371 |
| HAB01_H6 | 3370 | 0.7547 |
| HAB01_H7 | 2766 | 0.7601 |
| HAB01_H8 | 2751 | 1.236 |
| Lab01-huFc | 3002 | 0.6254 |
| JNJ7564-hIgG1 | 1436 | 26.95 |
| 5F11-hIgG1 | 2873 | 2.916 |
| anti-hel-hIgG1 | 194 | N/A |

TABLE 28
Binding activity of humanized antibody HAB02 for molp-8 cells

| | Maximum | EC50 |
|---|---|---|
| Antibody | value (MFI) | (nM) |
| HAB02_H1 | 1476 | 0.6023 |
| HAB02_H2 | 1395 | 0.7416 |
| HAB02_H3 | 1595 | 1.596 |
| HAB02_H4 | 1765 | 1.303 |
| HAB02_H5 | 1611 | 1.062 |
| HAB02_H6 | 1850 | 1.537 |
| HAB02_H7 | 1716 | 0.5474 |
| HAB02_H8 | 1686 | 1.064 |
| Lab02-huFc | 1614 | 0.8755 |
| JNJ7564-hIgG1 | 1287 | 41.25 |
| 5F11-hIgG1 | 2243 | 5.643 |
| anti-hel-hIgG1 | 211 | N/A |

TABLE 29
Binding activity of humanized antibody HAB03 for molp-8 cells

| | Maximum | EC50 |
|---|---|---|
| Antibody | value (MFI) | (nM) |
| HAB03_H5 | 2402 | 0.7375 |
| HAB03_H8 | 2061 | 1.218 |
| HAB03_H9 | 2281 | 0.849 |
| HAB03_H10 | 2381 | 0.7622 |
| HAB03_H11 | 2317 | 0.8122 |
| HAB03_H12 | 2721 | 0.6422 |
| Lab03-huFc | 2326 | 0.599 |
| JNJ7564-hIgG1 | 1408 | 34.43 |
| 5F11-hIgG1 | 3054 | 3.683 |
| anti-hel-hIgG1 | 261 | N/A |

TABLE 30
Binding activity of humanized antibody HAB04 for molp-8 cells

| | Maximum | EC50 |
|---|---|---|
| Antibody | value (MFI) | (nM) |
| HAB04_H3 | 751 | 3.037 |
| HAB04_H4 | 762 | 2.708 |
| HAB04_H5 | 696 | 2.797 |
| HAB04_H7 | 685 | 2.674 |
| HAB04_H8 | 783 | 1.742 |
| HAB04_H10 | 895 | 4.296 |
| HAB04_H14 | 837 | 1.352 |
| Lab04-huFc | 809 | 1.968 |
| JNJ7564-hIgG1 | 550 | 45.9 |
| 5F11-hIgG1 | 1733 | 12.58 |
| anti-hel-hIgG1 | 297 | N/A |

TABLE 31
Binding activity of humanized antibody
HAB01 for RPMI-8226 cells

| | Maximum | EC50 |
|---|---|---|
| Antibody | value (MFI) | (nM) |
| HAB01_H1 | 1115 | 103.1 |
| HAB01_H3 | 789 | 36.05 |
| HAB01_H6 | 712 | 25.73 |
| HAB01_H7 | 469 | 3.699 |
| HAB01_H8 | 389 | 1.775 |
| Lab01-huFc | 500 | 2.08 |
| JNJ7564-hIgG1 | 590 | ~5461 |
| 5F11-hIgG1 | 691 | 26.48 |
| anti-hel-hIgG1 | 237 | N/A |

TABLE 32
Binding activity of humanized antibody
HAB02 for RPMI-8226 cells

| | Maximum | EC50 |
|---|---|---|
| Antibody | value (MFI) | (nM) |
| HAB02_H1 | 344 | 3.716 |
| HAB02_H2 | 353 | 3.557 |
| HAB02_H3 | 300 | 1.881 |
| HAB02_H4 | 594 | 47.89 |
| HAB02_H5 | 359 | 1.758 |
| HAB02_H6 | 478 | 10.18 |
| HAB02_H7 | 325 | 0.2565 |
| HAB02_H8 | 339 | 1.504 |
| Lab02-huFc | 314 | 1.747 |
| JNJ7564-hIgG1 | 384 | ~635372 |
| 5F11-hIgG1 | 768 | 12.08 |
| anti-hel-hIgG1 | 164 | N/A |

TABLE 33
Binding activity of humanized antibody
HAB03 for RPMI-8226 cells

| | Maximum | EC50 |
|---|---|---|
| Antibody | value (MFI) | (nM) |
| HAB03_H5 | 469 | 41.52 |
| HAB03_H8 | 272 | 0.6603 |
| HAB03_H9 | 353 | 0.7775 |
| HAB03_H10 | 438 | 1.426 |
| HAB03_H11 | 348 | 0.6274 |
| HAB03_H12 | 681 | 51.06 |
| Lab03-huFc | 353 | 0.6032 |
| JNJ7564-hIgGI | 441 | ~1360606 |
| 5F11-hIgG1 | 672 | 18.69 |
| anti-hel-hIgG1 | 119 | N/A |

TABLE 34
Binding activity of humanized antibody
HAB04 for RPMI-8226 cells

| | Maximum | EC50 |
|---|---|---|
| Antibody | value (MFI) | (nM) |
| HAB04_H3 | 736 | 2.812 |
| HAB04_H4 | 737 | 2.294 |
| HAB04_H5 | 829 | 2.048 |
| HAB04_H7 | 781 | 2.419 |
| HAB04_H8 | 720 | 1.677 |
| HAB04_H10 | 861 | 3.052 |
| HAB04_H14 | 849 | 1.637 |
| Lab04-huFc | 843 | 2.494 |
| JNJ7564-hIgG1 | 654 | 73.42 |
| 5F11-hIgG1 | 1613 | 7.066 |
| anti-hel-hIgG1 | 229 | N/A |

### Example 6. Preparation of Vicugna pacos Nanobodies Against GPRC5D

### 6.1 Animal Immunization and Serum Titer Assay

One Vicugna pacos (alpaca) of 2-3 years old was selected, and 10 mL of blood was collected before immunization and used as the negative control serum. The Vicugna pacos was immunized by using CHO-K1-human GPRC5D overexpression cell line, the amount of each cell immunization was 2×10″ cells, and the immunization was performed once every 2 weeks. Animal serum was collected 7 days after each immunization for FACS assay of serum titers. The results of serum titers for the fourth immunization are shown in Table 35, in which the data are MFI values.

TABLE 35
FACS assay results of scrum of fourth immunization with GPRC5D

| | | | | HEK293- | HEK293- | HEK293- |
|---|---|---|---|---|---|---|
| | HEK293 | HEK293 | HEK293 | GPRC5D | GPRC5D | GPRC5D |
| Dilution | Negative | Third | Fourth | Negative | Third | Fourth |
| ratio | serum | immunization | immunization | serum | immunization | immunization |
| 1:100 | 503.42 | 1526.81 | 1582.99 | 460.85 | 2056.64 | 3952.00 |
| 1:1000 | 498.41 | 850.11 | 872.97 | 350.31 | 980.95 | 1100.1 |

### 6.2 Construction of Nanobody Phage Library

After the immunization was completed, 50 mL of peripheral blood was collected, and PBMCs were isolated using a lymphocyte isolation medium. The total RNA was extracted using the Trizol reagent, and cDNA was obtained according to the instructions of the reverse transcription kit Prime Script™ II 1st Strand cDNA Synthesis Kit (Takara, 6210A). The nanobody (VHH) fragments were amplified by nested PCR, and the phage library was constructed according to the method in Example 2.2.

### 6.3 Panning of Nanobodies Against GPRC5D

The phage library panning was performed using cells overexpressing human GPRC5D (293T-human GPRC5D) as the antigen, and the specific panning strategy is as follows: Cells were collected, washed twice with 5% serum-PBS, and immobilized with 4% paraformaldehyde (100 μL/well) at room temperature for 25-30 min. The phage dilution after blocking was added, and the mixture was incubated at 37° C. for 1 h. Unbound phages were removed, and the cells were washed 3 times with 0.1% PBST and 2 times with PBS. 500 μL of Gly-HCl eluent was added, and the mixture was incubated at 37° C. for 8 min to elute specifically-bound phages. The eluate was transferred into a 1.5 mL sterile centrifuge tube and quickly neutralized with 50 μL of Tris-HCl neutralization buffer. The phage supernatant was collected for the next round of biopanning. After multiple rounds of panning, the positive phages were continuously enriched in the panning process, so that nanobodies with good specificity and high affinity were selected.

### 6.4 Screening of Phage Clones Specifically Binding to GPRC5D

Single clones were selected from the plates of round 2 and round 3 and cultured in a 96-well plate. A 2YT medium containing an antibiotic and 1% glucose was added at 200 μL/well, and the mixture was cultured at 37° C. and 250 rpm overnight while shaking. 10 μL of the overnight culture was added to 100 μL of 2YT medium containing an antibiotic and 0.5% glucose, and the mixture was cultured until the OD₆₀₀ was 0.4-0.6. Helper phages were added at an infection ratio of 20:1, and the mixture was left to stand at 37° C. for 30 min. Then, the mixture was cultured at 37° C. for 30 min while shaking, 400 μL of 2YT medium containing an antibiotic was added, and the mixture was cultured at 30° C. overnight. The next day, the mixture was centrifuged at 5000 rpm and 4° C. for 20 min, and the resulting supernatant was used for ELISA assay and FACS assay of single clones.

293T-human GPRC5D and 293T negative cells were inoculated into a 96-well plate and cultured overnight. The next day, 100 μL of 4% paraformaldehyde was added to immobilize the cells. The plate was washed 2 times with PBS, 5% skimmed milk was added at 300 μL/well, and the mixture was blocked at 37° C. for 1 h. The phage culture solution supernatant and 5% skimmed milk were each added at 50 μL/well, and the mixture was incubated at 37° C. for 1 h. The plate was washed 5 times with PBST, and a horseradish peroxidase-labeled anti-M13 antibody (diluted with PBS according to 1:10000) was added. A TMB color development solution was added for color development, and OD_{450 nm} absorbance values were read using a microplate reader, and ELISA-positive clones were selected for FACS verification next.

ELISA-positive clones were further verified using the overexpression cell strain 293T-human GPRC5D, and the specific procedure was performed with reference to Example 2.4. Through multiple rounds of optimization and screening, 2 positive clones capable of recognizing GPRC5D were obtained and named Lab05 and Lab06, respectively. The CDRs of their sequences were analyzed using IMGT, KABAT, and Chothia numbering schemes. The corresponding sequence information is shown in Tables 36-37 below, wherein Table 36 shows the amino acid sequences of the 2 nanobody molecules, and Table 37 shows the results of IMGT, Kabat, and Chothia analysis of the CDRs of the 2 nanobody molecules.

TABLE 36
Amino acid sequences of anti-GPRCSD nanobodies

| | Sequence | |
|---|---|---|
| Antibody | No. | Amino acid sequence |
| Lab05 | SEQ ID NO: | QVQLVESGGGLVQPGGSLRLSCAASGILVSAYVMAWYRQP |
| | 76 | PGKQRDLVARLSTDGRTTYADSVKGRFTISKDNAKSTLYLQ |
| | | MNTLKSEDTAVYYCYAERSRGHWGQGTQVTVSS |
| Lab06 | SEQ ID NO: | QVQLVESGGGLVQPGGSLRLSCTASGFTFSSAWMYWFRQA |
| | 77 | PGKGLEWVSHIDPRGGSTYYADSVKGRFTISRDNAKNTLYL |
| | | QMRSLKSEDTAVYYCAKDLRGLGRGQGTQVTVSS |

TABLE 37
Analysis of CDR sequences of GPRCSD nanobodies by IMGT, KABAT, and
IMGT Analysis

| | Sequence | | Sequence | | Sequence | |
|---|---|---|---|---|---|---|
| Antibody | No. | CDR1 | No. | CDR2 | No. | CDR3 |
| Lab05 | SEQ ID | GILVSAY | SEQ ID | LSTDGRT | SEQ ID | YAERSRGH |
| | NO: 78 | V | NO: 79 | | NO: 80 | |
| Lab06 | SEQ ID | GFTFSSA | SEQ ID | IDPRGGST | SEQ ID | AKDLRGLG |
| | NO: 81 | W | NO: 82 | | NO: 83 | |

Kabat analysis

| | Sequence | | Sequence | | Sequence | |
|---|---|---|---|---|---|---|
| Antibody | No. | CDR1 | No. | CDR2 | No. | CDR3 |
| Lab05 | SEQ ID | AYVMA | SEQ ID | RLSTDGRTT | SEQ ID | ERSRGH |
| | NO: 84 | | NO: 85 | YADSVKG | NO: 86 | |
| Lab06 | SEQ ID | SAWMY | SEQ ID | HIDPRGGST | SEQ ID | DLRGLG |
| | NO: 87 | | NO: 88 | YYADSVKG | NO: 89 | |

Chothia Analysis

| | Sequence | | Sequence | | Sequence | |
|---|---|---|---|---|---|---|
| Antibody | No. | CDR1 | No. | CDR2 | No. | CDR3 |
| Lab05 | SEQ ID | GILVSAY | SEQ ID | STDGR | SEQ ID | ERSRGH |
| | NO: 90 | | NO: 91 | | NO: 86 | |
| Lab06 | SEQ ID | GFTFSSA | SEQ ID | DPRGGS | SEQ ID | DLRGLG |
| | NO: 92 | | NO: 93 | | NO: 89 | |

### Example 7. Humanization of Vicugna pacos Nanobodies

### 7.1 Humanization Design for Nanobodies

By alignment with the IMGT database (http://imgt.cines.fr) for human antibody variable region germline gene sequences, germline genes, with high homology to the nanobodies, from heavy chain variable regions were selected as templates, and CDR sequences of the nanobodies based on the IMGT or KABAT naming method were separately grafted into corresponding humanization templates to form variable region sequences in the order of FR1-CDR1-FR2-CDR2-FR3-CDR3-FR4.

The CDR sequences of the Lab05 antibody were defined based on the IMGT scheme, and the humanization templates thereof were IGHV3-74*03 and IGHJ3*01. The Lab05 antibody was renamed HAB05 during the humanization and named HABOS.H after CDR grafting.

The CDR sequences of the Lab06 antibody were defined based on the Kabat scheme, and the humanization templates thereof were IGHV3-23*04 and IGHJ3*01. The Lab06 antibody was renamed HAB06 during the humanization and named HAB06.H after CDR grafting.

CDRs of the antibodies HAB05 and HAB06 were respectively grafted into the humanization templates thereof to obtain humanized antibodies. The amino acid sequences of the humanization templates thereof are shown in Table 38.

TABLE 38
Specific amino acid sequences of humanization templates and humanized
antibodies

| | Sequence | |
|---|---|---|
| Sequence | No. | Amino acid sequence |
| IGHV3-74*03 | SEQ ID | EVQLVESGGGLVQPGGSLRLSCAASGFTFSSYWMHWVRQ |
| template | NO: 94 | APGKGLVWVSRINSDGSSTTYADSVKGRFTISRDNAKNTL |
| | | YLQMNSLRAEDTAVYYCAR |
| IGHV3-23*04 | SEQ ID | EVQLVESGGGLVQPGGSLRLSCAASGFTFSSYAMSWVRQA |
| template | NO: 95 | PGKGLEWVSAISGSGGSTYYADSVKGRFTISRDNSKNTLY |
| | | LQMNSLRAEDTAVYYCAK |
| IGHJ3*01 | SEQ ID | WGQGTMVTVSS |
| template | NO: 48 | |

### 7.2. Back Mutation Design for HABOS Humanized Antibodies

Key amino acids in the FR sequence of the HAB05 humanized antibody were back-mutated to amino acids corresponding to the Vicugna pacos antibody to ensure the original affinity. Details of the mutation points of the specific back mutation (back mutation points were numbered in a natural order) and the specific amino acid sequences are shown in Tables 39-40.

TABLE 39
Mutation points of back mutation of HAB05 humanized antibodies

| Antibody | Mutation point |
|---|---|
| HAB05-H2 | H35A, V37Y, G44Q, L45R, W47L, R71K |
| Graft(IGHV3-74*03) | |
| HAB05-H3 | H35A, V37Y, G44Q, L45R, W47L, R71K, N76S |
| Graft(IGHV3-74*03) | |

Note:
Graft(IGHV3-74*03) denotes that the CDRs of the nanobody are grafted into the human germline FR sequences; H35A denotes that H at position 35 of Graft(IGHV3-74*03) is mutated to A, and the same applies to others.

TABLE 40
Amino acid sequences of HAB05 humanized antibodies after back mutation

| Antibody | Sequence No. | Amino acid sequence |
|---|---|---|
| HAB05-H2 | SEQ ID NO: | EVQLVESGGGLVQPGGSLRLSCAASGILVSAYVMAWYRQA |
| | 96 | PGKQRVLVSRLSTDGRTTYADSVKGRFTISKDNAKNTLYL |
| | | QMNSLRAEDTAVYYCYAERSRGHWGQGTMVTVSS |
| HAB05-H3 | SEQ ID NO: | EVQLVESGGGLVQPGGSLRLSCAASGILVSAYVMAWYRQA |
| | 97 | PGKQRVLVSRLSTDGRTTYADSVKGRFTISKDNAKSTLYLQ |
| | | MNSLRAEDTAVYYCYAERSRGHWGQGTMVTVSS |

The results of the IMGT numbering scheme-based CDR analysis of HAB05 humanized antibodies are shown in Table 41:

TABLE 41
Results of IMGT numbering scheme-based CDR analysis of HAB05 humanized
antibodies

| | Sequence | | Sequence | | Sequence | CDR3 |
|---|---|---|---|---|---|---|
| Antibody | No. | CDR1 | No. | CDR2 | No. | |
| HAB05-H2 | SEQ ID | GILVSAY | SEQ ID | LSTDGRT | SEQ ID | YAERSRGH |
| HAB05-H3 | NO: 78 | V | NO: 79 | | NO: 80 | |

### 7.3. Back Mutation Design for HAB06 Humanized Antibodies

Key amino acids in the FR sequence of the HAB06 humanized antibody were back-mutated to amino acids corresponding to the Vicugna pacos antibody to ensure the original affinity. Details of the mutation points of the specific back mutation (back mutation points were numbered in a natural order) and the specific amino acid sequences are shown in Tables 42-43.

TABLE 42
Mutation points of back mutation of HAB06 humanized antibodies

| Antibody | Mutation point |
|---|---|
| HAB06-H1 | V37F, W105R |
| Graft(IGHV3-23*04) | |
| HAB06-H5 | V37F, S75A, W10SR, M110Q |
| Graft(IGHV3-23*04) | |

Note: Graft (IGHV3-23*04) denotes that the CDRs of the nanobody are grafted into the human germline FR sequences; V37F denotes that V at position 37 of Graft (IGHV3-23*04) is mutated to F, and the same applies to others.

TABLE 43
Amino acid sequences of HAB06 humanized antibodies after back mutation

| | Sequence | |
|---|---|---|
| Antibody | No. | Amino acid sequence |
| HAB06-H1 | SEQ ID NO: | EVQLVESGGGLVQPGGSLRLSCAASGFTFSSAWMYWFRQA |
| | 98 | PGKGLEWVSHIDPRGGSTYYADSVKGRFTISRDNSKNTLYL |
| | | QMNSLRAEDTAVYYCAKDLRGLGRGQGTMVTVSS |
| HAB06-H5 | SEQ ID NO: | EVQLVESGGGLVQPGGSLRLSCAASGFTFSSAWMYWFRQA |
| | 99 | PGKGLEWVSHIDPRGGSTYYADSVKGRFTISRDNAKNTLY |
| | | LQMNSLRAEDTAVYYCAKDLRGLGRGQGTQVTVSS |

The results of the Kabat numbering scheme-based CDR analysis of HAB06 humanized antibodies are shown in Table 44:

TABLE 44
Results of Kabat numbering scheme-based CDR analysis of HAB06 humanized
antibodies

| | Sequence | | Sequence | | Sequence | |
|---|---|---|---|---|---|---|
| Antibody | No. | CDR1 | No. | CDR2 | No. | CDR3 |
| HAB06-H1 | SEQ ID | SAWMY | SEQ ID | HIDPRGGSTY | SEQ ID | DLRGLG |
| HAB06-HS | NO: 87 | | NO: 88 | YADSVKG | NO: 89 | |

### Example 8. Expression, Purification and Binding Assay of Chimeric and Humanized Nanobodies

### 8.1 Expression and Purification of Nanobodies

The nanobody sequences obtained after screening, the humanization sequences, and the control antibody sequence were each cloned into a eukaryotic expression vector pTT5 with a hIgG1-Fc tag. Expi293F cells (Gibco, A14527) were transiently transfected with the vector by using PEI, cultured for 7 days, and then centrifuged at high speed to collect the cell culture supernatant expressing the antibodies. The antibodies were purified according to the purification method described in Example 1.2 to obtain corresponding recombinant nanobodies and humanized nanobodies. The recombinant nanobodies were named Lab05-huFc and Lab06-huFc, respectively. The humanized nanobodies were named HABOS-H2, HAB05-H3, HAB06-H1, and HAB06-H5, respectively. The positive control sequence was derived from Patent No. WO 2020/092854 A2 and named MCARH109-scFv-huFc, and its VH and VL sequences are shown in Table 45. The purified antibodies were each subjected to purity detection using SEC-HPLC and then aliquoted for later use after being determined to be acceptable.

TABLE 45
Control antibody sequences for binding assay of chimeric and humanized
nanobodies

| Sequence | Sequence No. | Amino acid sequence |
|---|---|---|
| MCARH109- | SEQ ID NO: 100 | QVQLVESGGGLVHPGGSLRLSCAASGFTFRSHSMNWV |
| VH | | RQAPGKGLEWVSSISSDSTYTYYADSVKGRFTISRDNA |
| | | KNSLYLQMNSLRAEDTAVYYCARSGGQWKYYDYWG |
| | | QGTLVTVSS |
| MCARH109- | SEQ ID NO: 101 | SSELTQDPAVSVALGQTVRITCQGDSLRSYYASWYQQK |
| VL | | PGQAPVLVIYGKNNRPSGIPDRFSGSSSGNTASLTITGA |
| | | QAEDEADYYCNSRDSSGNPPVVFGGGTKLTVL |

### 8.2 Assay on Binding of Antibodies to Cells Overexpressing Human GPRC5D by Flow Cytometry Assay (FACS)

CHOK1-human GPRC5D was expanded to the logarithmic growth phase in a T-175 culture flask, the medium was removed by pipetting, and the cells were washed 2 times with a PBS buffer and digested with trypsin. Then, a complete medium was added to stop the digestion, and the cells were pipetted to form a single cell suspension. After counting, the cells were centrifuged, and the cell pellet was resuspended with an FACS buffer (PBS+2% fetal bovine serum) to 2×10⁶ cells/mL. Then, FACS assay and data analysis were performed with reference to the method in Example 3.2. The secondary antibody used was AlexaFluor®647 AffiniPure Goat Anti-Human IgG (H+L)-labeled secondary antibody (Jackson, 109-605-003). The results are shown in FIG. 8. The recombinant antibodies Lab05-huFc and Lab06-huFc as well as the humanized antibodies all can efficiently bind to overexpression cells CHOK1-human GPRC5D. The specific binding activity and values are shown in Table 46 below.

TABLE 46
Binding activity of recombinant and humanized
antibodies for CHOK1-human GPRCSD cells

| | Maximum | EC50 |
|---|---|---|
| Antibody | value (MFI) | (nM) |
| Lab05-huFc | 215063 | 0.2791 |
| HAB05-H2 | 227537 | 0.5139 |
| HAB05-H3 | 210706 | 0.2485 |
| Lab06-huFc | 201140 | 0.2203 |
| HAB06-H1 | 189248 | 0.3244 |
| HAB06-H5 | 187264 | 0.2406 |
| MCARH109-scFv-huFc | 197827 | 0.2862 |
| anti-hel-hIgG1 | 1487 | N/A |

### 8.3 Assay on Binding of Humanized Antibodies to Multiple Myeloma Cells by Flow Cytometry Assay (FACS)

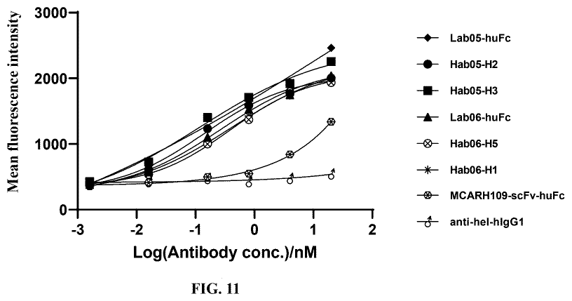

The preparation of the assay cells and the antibodies to be tested and the assay were performed with reference to Example 3.2. The assay results are shown in FIGS. 9-11. The recombinant antibodies and the humanized antibodies all have good binding activity for endogenous cells NCI-H929, molp-8, and RPMI-8226, and specific binding activity and values are shown in Tables 47-49 below.

TABLE 47
Binding activity of recombinant and humanized
antibodies for endogenous cell NCI-H929

| | Maximum | EC50 |
|---|---|---|
| Antibody | value (MFI) | (nM) |
| Lab05-huFc | 30373 | 0.1107 |
| HAB05-H2 | 28953 | 0.2017 |
| HAB05-H3 | 29233 | 0.08613 |
| Lab06-huFc | 34254 | 0.2727 |
| HAB06-H1 | 30635 | 0.1656 |
| HAB06-H5 | 29654 | 0.3803 |
| MCARH109-scFv-huFc | 5684 | 2.452 |
| anti-hel-hIgG1 | 369 | N/A |

TABLE 48
Binding activity of recombinant and humanized
antibodies for endogenous cell molp-8

| | Maximum | EC50 |
|---|---|---|
| Antibody | value (MFI) | (nM) |
| Lab05-huFc | 14926 | 0.06161 |
| HAB05-H2 | 14701 | 0.08391 |
| HAB05-H3 | 14678 | 0.0433 |
| Lab06-huFc | 15851 | 0.1317 |
| HAB06-H1 | 15025 | 0.1403 |
| HAB06-H5 | 13893 | 0.1784 |
| MCARH109-scFv-huFc | 2762 | 3.232 |
| anti-hel-hIgG1 | 586 | N/A |

TABLE 49
Binding activity of recombinant and humanized
antibodies for endogenous cell RPMI-8226

| | Maximum | EC50 |
|---|---|---|
| Antibody | value (MFI) | (nM) |
| Lab05-huFc | 2464 | 31.62 |
| HAB05-H2 | 2004 | 0.1406 |
| HAB05-H3 | 2256 | 0.1202 |
| Lab06-huFc | 2041 | 0.2608 |
| HAB06-H1 | 2010 | 0.4064 |
| HAB06-H5 | 1936 | 0.3872 |
| MCARH109-scFv-buFc | 1339 | N/A |
| anti-hel-hIgG1 | 543 | N/A |

1. An antibody or an antigen-binding fragment thereof that specifically binds to human or monkey GPRC5D, comprising a CDR1, a CDR2, and a CDR3, wherein the CDR1, the CDR2, and the CDR3 comprise an HCDR1, an HCDR2, and an HCDR3, respectively, of a VHH domain set forth in any one of SEQ ID NOs: 10-13, 50-75, 76-77, and 96-99.

2. The antibody or the antigen-binding fragment thereof according to claim 1, wherein the HCDR1, the HCDR2, and the HCDR3 are determined according to IMGT, Kabat, or Chothia numbering scheme and are selected from any one of the following:
(1) based on the IMGT, Kabat, or Chothia numbering scheme, the HCDR1, the HCDR2, and the HCDR3 comprise the amino acid sequences of SEQ ID NOs: 14, 15, and 16, respectively, the amino acid sequences of SEQ ID NOs: 26, 27, and 28, respectively, or the amino acid sequences of SEQ ID NOs: 38, 39, and 28, respectively;
(2) based on the IMGT, Kabat, or Chothia numbering scheme, the HCDR1, the HCDR2, and the HCDR3 comprise the amino acid sequences of SEQ ID NOs: 17, 18, and 19, respectively, the amino acid sequences of SEQ ID NOs: 29, 30, and 31, respectively, or the amino acid sequences of SEQ ID NOs: 40, 41, and 31, respectively;
(3) based on the IMGT, Kabat, or Chothia numbering scheme, the HCDR1, the HCDR2, and the HCDR3 comprise the amino acid sequences of SEQ ID NOs: 20, 21, and 22, respectively, the amino acid sequences of SEQ ID NOs: 32, 33, and 34, respectively, or the amino acid sequences of SEQ ID NOs: 42, 43, and 34, respectively;
(4) based on the IMGT, Kabat, or Chothia numbering scheme, the HCDR1, the HCDR2, and the HCDR3 comprise the amino acid sequences of SEQ ID NOs: 23, 24, and 25, respectively, the amino acid sequences of SEQ ID NOs: 35, 36, and 37, respectively, or the amino acid sequences of SEQ ID NOs: 44, 45, and 37, respectively;
(5) based on the IMGT, Kabat, or Chothia numbering scheme, the HCDR1, the HCDR2, and the HCDR3 comprise the amino acid sequences of SEQ ID NOs: 78, 79, and 80, respectively, the amino acid sequences of SEQ ID NOs: 84, 85, and 86, respectively, or the amino acid sequences of SEQ ID NOs: 90, 91, and 86, respectively;
(6) based on the IMGT, Kabat, or Chothia numbering scheme, the HCDR1, the HCDR2, and the HCDR3 comprise the amino acid sequences of SEQ ID NOs: 81, 82, and 83, respectively, the amino acid sequences of SEQ ID NOs: 87, 88, and 89, respectively, or the amino acid sequences of SEQ ID NOs: 92, 93, and 89, respectively; or
(7) amino acid sequences having 1, 2, or 3 mutations compared with the HCDR1, the HCDR2, and/or the HCDR3 described in (1)-(6), wherein the mutation is selected from an insertion, a deletion, and/or a substitution.

3. The antibody or the antigen-binding fragment thereof according to claim 1, wherein the CDR1, the CDR2, and/or the CDR3 comprise amino acid sequences having at least 80, 85%, 90%, 91%, 92%, 93%, 94%, 95%, 96%, 97%, 98%, 99%, or 100% identity to the HCDR1, the HCDR2, and/or the HCDR3, respectively, according to claim 1.

4. The antibody or the antigen-binding fragment thereof according to claim 1, wherein the antibody or the antigen-binding fragment thereof is a single domain antibody, and
the single domain antibody comprises the amino acid sequence set forth in any one of SEQ ID NOs: 10-13, 50-75, 76-77, and 96-99; or
the single domain antibody comprises an amino acid sequence having at most 20, 19, 18, 17, 16, 15, 14, 13, 12, 11, 10, 9, 8, 7, 6, 5, 4, 3, 2, or 1 mutation compared with the sequence set forth in any one of SEQ ID NOs: 10-13, 50-75, 76-77, and 96-99, wherein the mutation is selected from an insertion, a deletion, and/or a substitution; or
the single domain antibody comprises an amino acid sequence having at least 80%, 85%, 90%, 91%, 92%, 93%, 94%, 95%, 96%, 97%, 98%, 99%, or 100% identity to the sequence set forth in any one of SEQ ID NOs: 10-13, 50-75, 76-77, and 96-99.

5. (canceled)

6. The antibody or the antigen-binding fragment thereof according to claim 1, wherein the antibody or the antigen-binding fragment thereof is: (1) a chimeric antibody or a fragment thereof, (2) a humanized antibody or a fragment thereof, or (3) a fully human antibody or a fragment thereof.

7. The antibody or the antigen-binding fragment thereof according to claim 1, wherein the antibody or the antigen-binding fragment thereof comprises or does not comprise an antibody heavy chain constant region, and the antibody heavy chain constant region is selected from human, Vicugna pacos, mouse, rat, rabbit, and sheep.

8. The antibody or the antigen-binding fragment thereof according to claim 1, wherein the antibody or the antigen-binding fragment thereof is further conjugated to a therapeutic agent or a tracer, and the therapeutic agent is selected from a radioisotope, a chemotherapeutic agent, an immunomodulator, and combinations thereof, and the tracer is selected from a radiocontrast medium, a paramagnetic ion, a metal, a fluorescent label, a chemiluminescent label, an ultrasound contrast agent, a photosensitizer, and combinations thereof; and/or wherein the antibody or the antigen-binding fragment thereof is further linked with an additional functional molecule, and the additional functional molecule is selected from one or more of a signal peptide, a protein tag, a cytokine, an angiogenesis inhibitor, and an immune checkpoint inhibitor.

9. (canceled)

10. A multispecific antigen-binding molecule, comprising the antibody or the antigen-binding fragment thereof according to claim 1 and an antigen-binding molecule binding to an antigen other than GPRC5D or binding to a GPRC5D epitope different from that of the antibody or the antigen-binding fragment thereof according to claim 1, wherein the antigen other than GPRC5D is selected from: CD3, CD16, CD137, CD258, PD-1, PD-L1, 4-1BB, CD40, CD64, EGFR, VEGF, HER2, HER1, HER3, IGF-1R, phosphatidylserine (PS), C-Met, HSA, BCMA, MSLN, blood-brain barrier receptor, GPC3, PSMA, CD33, GD2, ROR1, ROR2, FRα, and Gucy2C;
and wherein the antigen-binding molecule binding to an antigen other than GPRC5D is an antibody or an antigen-binding fragment thereof.

11. An isolated nucleic acid fragment encoding the antibody or the antigen-binding fragment thereof according to claim 1.

12. A vector, comprising the nucleic acid fragment according to claim 11.

13. A host cell, comprising the isolated nucleic acid fragment according to claim 11.

14. (canceled)

15. A pharmaceutical composition, comprising: the antibody or the antigen-binding fragment thereof according to claim 1, and a pharmaceutically acceptable carrier, a diluent, or an adjuvant.

16. A method for treating a tumor or cancer, comprising administering to a subject an effective amount of the antibody or the antigen-binding fragment thereof according to claim 1.

17. (canceled)

18. (canceled)

19. A kit, comprising: the antibody or the antigen-binding fragment thereof according to claim 1.

20. A method for detecting expression of GPRC5D in a biological sample, comprising contacting the biological sample with the antibody or the antigen-binding fragment thereof according to claim 1 under conditions that allow the antibody or the antigen-binding fragment thereof and GPRC5D to form a complex, and detecting formation of the complex and indicating presence or an expression level of GPRC5D in the sample.

21. (canceled)

22. The antibody or the antigen-binding fragment thereof according to claim 2, wherein the substitution is a conservative amino acid substitution.

23. The antibody or the antigen-binding fragment thereof according to claim 4, wherein the substitution is a conservative amino acid substitution.

24. The host cell according to claim 13, wherein the cell is a prokaryotic cell or a eukaryotic cell.

25. The host cell according to claim 13, wherein the cell is a bacterium, a fungus, an insect cell, or a mammalian cell.

26. The method according to claim 16, wherein the tumor or cancer is B cell lymphoma or multiple myeloma (MM).
